(19) United States
(12) Patent Application Publication
(10) Pub. No.: US 2023/0364032 A1
(43) Pub. Date: Nov. 16, 2023

(54) USE OF 2-HOBA TO TREAT ATHEROSCLEROSIS

(71) Applicant: Vanderbilt University, Nashville, TN (US)

(72) Inventors: MacRae F. Linton, Nashville, TN (US); Sean S. Davies, Nashville, TN (US); Olivier Boutaud, Nashville, TN (US)

(21) Appl. No.: 17/928,765

(22) PCT Filed: Jun. 1, 2021
(86) PCT No.: PCT/US21/35314

§ 371 Date: Nov. 30, 2022

Related U.S. Application Data
(60) Provisional application No. 63/033,127, filed on Jun. 1, 2020.

(51) Int. Cl. A61K 31/135; A61P 9/10

(52) U.S. Cl. CPC A61K 31/135; A61P 9/10

(57) ABSTRACT

A method of treating familial hypercholesterolemia accelerated atherosclerosis in a subject in need thereof, comprising administering an effective amount of a dicarbonyl scavenger.

### GOVERNMENT SUPPORT

This invention was made with government support under HL116263 and DK59637 awarded by the NIH. The government has certain rights to this invention.

### BACKGROUND OF THE INVENTION

Atherosclerosis, the underlying cause of heart attack and stroke, is the most common cause of death and disability in the industrial world 1. Elevated levels of apolipoprotein B (LDL and VLDL) containing lipoproteins and low levels of HDL increase the risk of atherosclerosis¹. Although lowering LDL with HMG-CoA reductase inhibitors has been shown to reduce the risk of heart attack and stroke in large outcomes trials, substantial residual risk for cardiovascular events remains². Atherosclerosis is a chronic inflammatory disease with oxidative stress playing a critical role^{3,4}. Oxidative modification of apoB containing lipoproteins enhances internalization leading to foam cell formation^{1, 5}. In addition, oxidized LDL induces inflammation, immune cell activation, and cellular toxicity^{1, 5}. HDL protects against atherosclerosis via multiple roles including promoting cholesterol efflux, preventing LDL oxidation, maintaining endothelial barrier function, and by minimizing cellular oxidative stress and inflammation^{1, 4, 6}. HDL-C concentration is inversely associated with cardiovascular disease (CVD)⁶, but recent studies suggest that assays of HDL function may provide new independent markers for CVD risk^{7, 8}. Evidence has mounted that oxidative modification of HDL compromises its functions, and studies suggest that oxidized HDL is indeed proatherogenic^{1, 6, 9}.

During lipid peroxidation, highly reactive dicarbonyls, including 4-oxo-nonenal (4-ONE) malondialdehyde (MDA) and isolevuglandins (IsoLGs) are formed. These reactive lipid dicarbonyls covalently bind to DNA, proteins, and phospholipid causing alterations in lipoprotein and cellular functions^{1, 10, 11}. In particular, modification with reactive lipid dicarbonyls promotes inflammatory responses and toxicity that may be relevant to atherosclerosis^{12, 13, 14, 15}. Identifying effective strategies to assess the contribution of reactive lipid dicarbonyls to disease processes in vivo has been challenging. Although formation of reactive lipid species, including dicarbonyls, theoretically could be suppressed simply by lowering levels of reactive oxygen species (ROS) using dietary antioxidants, the use of antioxidants to prevent atherosclerotic cardiovascular events has proven problematic with most clinical outcomes trials failing to show a benefit^{1, 16}. Dietary antioxidants like vitamin C and vitamin E are relatively ineffective suppressors of oxidative injury and lipid peroxidation. In fact, careful studies of patients with hypercholesterolemia found that the doses of vitamin E required to significantly reduce lipid peroxidation were substantially greater than those typically used in most clinical trials 17. Furthermore, the high doses of antioxidants needed to suppress lipid peroxidation have been associated with significant adverse effects, likely because ROS play critical roles in normal physiology, including protection against bacterial infection and in a number of cell signaling pathways. Finally, for discovery purposes, the use of antioxidants provides little information about the role of reactive lipid dicarbonyls because suppression of ROS inhibits formation of a broad spectrum of oxidatively modified macromolecules in addition to reactive lipid dicarbonyl species.

An alternative approach to broad suppression of ROS utilizing antioxidants is to use small molecule scavengers that selectively react with lipid dicarbonyl species without altering ROS levels, thereby preventing reactive lipid dicarbonyls from modifying cellular macromolecules without disrupting normal ROS signaling and function. 2-hydroxybenzylamine (2-HOBA) rapidly reacts with lipid dicarbonyls such as IsoLG, ONE, and MDA, but not with lipid monocarbonyls such as 4-hydroxynonenal^{15, 18, 19, 20} The 2-HOBA isomer 4-hydroxybenzylamine (4-HOBA) is ineffective as a dicarbonyl scavenger²¹. Both of these compounds are orally bioavailable, so they can be used to examine the effects of lipid dicarbonyl scavenging in vivo^{13, 22}. 2-HOBA protects against oxidative stress associated hypertension¹³, oxidant induced cytotoxicity¹⁵, neurodegeneration,¹⁴ and rapid pacing induced amyloid oligomer formation²³. While there is evidence that reactive lipid dicarbonyls play a role in atherogenesis^{6, 7}, to date the effects of scavenging lipid dicarbonyl on the development of atherosclerosis have not been examined.

The present inventors have discovered that treatment with compounds of the present invention significantly attenuates atherosclerosis development. The present inventors have discovered that compound of the present invention inhibit cell death and necrotic core formation in lesions, leading to the formation of characteristics of more stable plaques as evidenced by increased lesion collagen content and fibrous cap thickness. Consistent with the decrease in atherosclerosis from 2-HOBA treatment being due to scavenging of reactive dicarbonyls, the atherosclerotic lesion MDA and IsoLG adduct content was markedly reduced in 2-HOBA treated versus control mice. The present inventors further show that treatment with compounds of the present invention results in decreased MDA-LDL and MDA-HDL. In addition, MDA-apoAI adduct formation was decreased, and importantly, 2-HOBA treatment caused more efficient HDL function in reducing macrophage cholesterol stores. Thus, scavenging of reactive carbonyls with compounds of the present invention has multiple antiatherogenic therapeutic effects that likely contribute to its ability to reduce the development of atherosclerosis.

The present inventors have also discovered that HDL from humans with severe familial hypercholesterolemia (FH) contained increased MDA adducts versus control subjects, and that FH-HDL were extremely impaired in reducing macrophage cholesterol stores. Thus, one embodiment of the present invention is reactive dicarbonyl scavenging in a subject in need thereof as a novel therapeutic approach to prevent and treat human atherosclerosis.

### INTRODUCTION AND SUMMARY OF THE INVENTION

The present inventors have shown that the pathogenesis of atherosclerosis may be accelerated by oxidative stress, which produces lipid peroxidation. Among the products of lipid peroxidation are highly reactive dicarbonyls including isolevuglandins (IsoLGs) and malondialdehyde (MDA) that covalently modify proteins. Embodiments of the present invention include treatment with compounds of the present invention, including the dicarbonyl scavenger, 2-hydroxybenzylamine (2-HOBA, salicylamine) on HDL function and atherosclerosis in hyperlipidemic Ldlr^{-/-} mice, a model of familial hypercholesterolemia (FH).

Compared to mice treated with vehicle, 2-HOBA significantly decreased atherosclerosis in hypercholesterolemic Ldlr^{-/-} mice by 31% in the proximal aortas and 60% in en face aortas, in the absence of changes in blood lipid levels. 2-HOBA reduced MDA content in HDL and LDL. Consuming a western diet increased plasma MDA-apoAI adduct levels in Ldlr^{-/-} mice. 2-HOBA inhibited MDA-apoAI formation and increased the capacity of the mouse HDL to reduce macrophage cholesterol stores.

The present inventors also show that 2-HOBA reduced the MDA- and IsoLG-lysyl content in atherosclerotic aortas in Ldlr^{-/-} mice. Furthermore, 2-HOBA diminished oxidative stress-induced inflammatory responses in macrophages, reduced the number of TUNEL-positive cells in atherosclerotic lesions by 72%, and decreased serum proinflammatory cytokines. Furthermore, 2-HOBA enhanced efferocytosis and promoted characteristics of stable plaque formation in mice as evidenced by a 69% (p<0.01) reduction in necrotic core and by increased collagen content (2.7-fold) and fibrous cap thickness (2.1-fold). HDL from patients with FH had increased MDA content resulting in a reduced ability of FH-HDL to decrease macrophage cholesterol content versus controls. The present invention shows that dicarbonyl scavenging with 2-HOBA has multiple atheroprotective effects on lipoproteins and reduces atherosclerosis in a murine model of FH, supporting its potential as a novel therapeutic approach for the prevention and treatment of human atherosclerotic cardiovascular disease.

One aspect of the present invention is a method of using compounds of the present invention to scavenge MDA.

Another aspect of the present invention is a method of protecting HDL and LDL from reactive dicarbonyls.

Another aspect of the present invention is a method of treating atherosclerosis in a subject in need thereof, comprising administering an effective amount of a dicarbonyl scavenger.

In some embodiments, the subject is diagnosed with familial hypercholesterolemia.

In some embodiments, the reactive dicarbonyl is isolevuglandins (IsoLGs) and malondialdehyde (MDA).

In some embodiments, the compound is selected from the following formula:

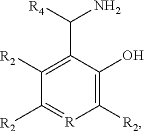

wherein R is C—R₂; each R₂ is independent and chosen from H, substituted or unsubstituted alkyl, halogen, alkyl, substituted or unsubstituted alkoxy, hydroxyl, nitro; R₄ is H, 2H, substituted or unsubstituted alkyl, carboxyl; and pharmaceutically acceptable salts thereof.

In some embodiments, the compound is selected from the following formula:

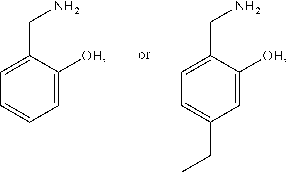

or a pharmaceutically acceptable salt thereof.

In some embodiments, the compound is 2-hydroxybenzylamine, ethyl-2-hydroxybenzylamine, or methyl-2-hydroxybenzylamine.

In some embodiments, the compound is selected from the following formula:

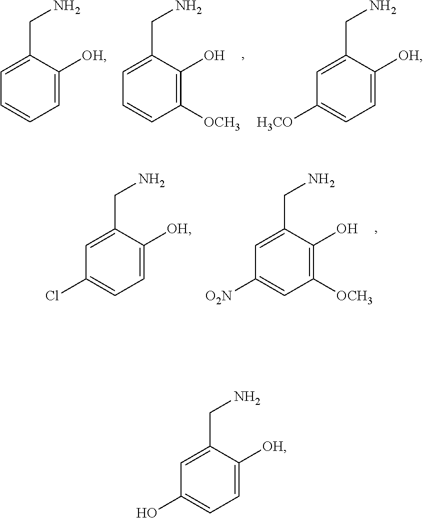

or a pharmaceutically acceptable salt thereof.

In other embodiments, the compound is chosen from:

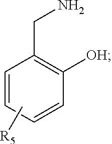

wherein R₅ is H, —CH₃, —CH₂CH₃, —CH(CH₃)—CH₃.

Another embodiment of the present invention is a method of reducing MDA- and IsoLG-lysyl content in atherosclerotic aortas in a subject in need thereof, comprising administering an effective dicarbonyl scavenging amount of a compound may be selected from the following formula:

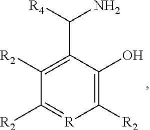

wherein R is C—R₂; each R₂ is independent and chosen from H, substituted or unsubstituted alkyl, halogen, alkyl, substituted or unsubstituted alkoxy, hydroxyl, nitro; R₄ is H, 2H, substituted or unsubstituted alkyl, carboxyl; and pharmaceutically acceptable salts thereof.

Another embodiment of the present invention is a method of treating atherosclerosis in a subject in need thereof, comprising administering an effective dicarbonyl scavenging amount of a compound of the following formula:

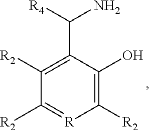

wherein R is C—R₂; each R₂ is independent and chosen from H, substituted or unsubstituted alkyl, halogen, alkyl, substituted or unsubstituted alkoxy, hydroxyl, nitro; R₄ is H, 2H, substituted or unsubstituted alkyl, carboxyl; and pharmaceutically acceptable salts thereof; and co-administering a drug with a known side effect of treating atherosclerosis.

### BRIEF DESCRIPTION OF THE FIGURES

FIG. 1A-1E show that 2-HOBA attenuates atherosclerosis in hypercholesterolemic female Ldlr^{-/-} mice. Specifically, 8-week old Ldlr^{-/-} mice were pretreated with 1 g/L 2-HOBA or 1 g/L 4-HOBA (nonreactive analogue) or vehicle (water) for 2 weeks and then treatment was continued for 16 weeks dueing which the mice were fed a Western diet. FIGS. A and C are representative images that show Oil-Red-O stain in proximal aorta root sections (FIG. A) and in open-pinned aortas (FIG. C). FIGS. B and D show quantitation of the mean Oil-Red-O stainable lesion area in aorta root sections (FIG. B) and en face aortas (FIG. D). FIG. E shows the plasma total cholesterol and triglyceride levels. (FIGS. B, D, and E) N=9 or 10 per group. **p<0.01, ***p, 0.001, One way ANOVA with Bonferroni's post hoc test.

FIG. 2A-2E show that 2-HOBA decreases the MDA adduct content of proximal aortic atherosclerotic lesions in Ldlr^{-/-} mice. MDA was detected by immunofluorsscence using anti-MDA primary antibody and fluorescent-labeled secondary antibody. Nuclei were counterstained with Hoechst (Blue). FIG. 2A shows representative images that show MDA staining (Red) in proximal aortic root sections. FIG. 2B shows quantitation of the mean MDA positive lesion area in aortic root sections using ImageJ software. Data are expressed as mean±SEM, N=6 per group, ***p, 0.001. One way ANOVA with Bonferroni's post hoc test. FIGS. C and D show aortic tissues isolated from the Ldlr^{-/-} mice and Dilysyl-MDA crosslinks (FIG. C) or IsoLG-Lysyl (FIG. D) were measured by LC/MS/MS. Data are presented as mean±SEM, (FIGS. C and D) N=8 or 9 per group, *p<0.05, Mann-Whotney test.

FIG. 3A-3D show 2-HOBA promotes features of stable atherosclerotic plaques in Ldlr^{-/-} mice. Masson's Trichrome stain was done to analyze characteristics associated with atherosclerotic lesion stability in proximal aorta sections of Ldlr^{-/-} mice. FIG. 3A shows representative images that show Masson's Trichrome stain in aorta root sections. The collagen content (FIG. 3A), figrous cap thickness (FIG. 3C) and necrotic area (FIG. D). were quantified using ImageJ software. N=8 per group. *p<0.05, One way ANOVA with Bonferroni's post hoc test. Scale bar=100 μm. Blue shows collagen, Red, cytoplasm, Black, nuclei.

FIGS. 4A-4D show that 2-HOBA prevents cell death and increases efferocytosis in atherosclerotic lesions of Ldlr^{-/-} mice. (4A) Representative images show dead cells that were detected by TUNEL staining (Red) of proximal aorta sections. Macrophages were detected by anti-macrophage primary antibody (green), and nuclei were counterstained with Hoechst (blue). (4B) A representative image taken at a higher magnification to indicate macrophage-associated TUNEL stain (yellow arrows) and white arrows indicate free dead cells that were not associated with macrophages. (4C) Quantitation of the number of TUNEL-positive nuclei in proximal aortic sections. (4D) Efferocytosis was examined by quantitating the free versus macrophage-associated TUNEL-positive cells in the proximal aortic sections. Data are expressed as mean±SEM (N=8 per group). Scale bar=50 **p<0.01, One-way ANOVA with Bonferroni's post hoc test.

FIGS. 5A-5D show that 2-HOBA reduces the plasma inflammatory cytokines in hypercholesterolemic Ldlr′⁻. mice. The inflammatory cytokines including IL-1β (5A), IL-6 (5B), TNF-α (5C) and SAA (5D) were measured by ELISA in plasma from mice consuming a western diet for 16 weeks and treated with 2-HOBA, 4-HOBA, or vehicle. N=8 per group.*p<0.05, **p<0.01. ***p<0.001. One-way ANOVA with Bonferroni's post hoc test.

FIG. 6A-H show that in vitro treatment with 2-HOBA suppresses oxidative stress-induced cell apoptosis and inflammation. (6A and 6B) Mouse aortic endothelial cells (6A) or primary macrophages (6B) were incubated for 24 h with 250 μM H₂O₂ alone or with either 4-HOBA or 2-HOBA (500 μM). Apoptotic cells were then detected by Annexin V staining and flow cytometry. (6C to 6H) The mRNA levels of IL-1β, IL-6, and TNF-α were analyzed by real time PCR in the peritoneal macrophages incubated for 24 h with either oxidized LDL (6C-6E) or 250 μM H₂O₂ (6F-6H) alone or with either 4-HOBA or 2-HOBA (500 μM). (6A to 6H) Data are presented as mean±SEM from three independent experiments, ***p<0.001, One-way ANOVA with Bonferroni's post hoc test.

FIGS. 7A-7G show the effects of 2-HOBA on MDA-HDL adducts and HDL function. (7A) The levels or MDA adducts were measured by ELISA in HDL isolated from Ldlr mice treated as described in FIG. 1. Data are presented as mean±SEM (N=8 pet group), *** p<0.001, One-way ANOVA with Bonferroni's post hoc test. (7B) Western blots of apoAl and MDA-apoAl in HDL isolated from plasma by immunoprecipitation using primary anti-apoAl antibody. Ldlf mice were treated as described In FIG. 1 and apoAl and MDA-apoAl from Ldlr mice consuming a chow diet are included f0t comparison. (7C) Quantitation using lmageJ software of the mean density ratio (arbitrary units) of MDA-apoAl to apoAl detected by Western blotting (7B). (7D The HDL was isolated from the plasma of Ld/r-mice consuming a western diet for 16 weeks and treated with 2-HOBA or 4-HOBA or vehicle. Cholesterol enriched macrophages were incubated for 24 h with HDL (25 μg protein/ml). and the % reduction in cellular cholesterol content measured. Data presented as mean±SEM. N=7 per group, *p<0.05.**p<0.01, One-way ANOVA with Bonferroni's post hoc test (7E) The MDA adducts were measured by ELISA in HDL isolated from control or FH subjects before and after LDL apheresis. N=7 or 8, ***p<0.001, One-way ANOVA with Bonferroni's post hoc test. (7F) The MDA-Lysyl crosslink content in HDL from control or FH subjects (n=6 per group), p=0.02, Mann-Whitney test (7G) The capacity of HDL from control or FH subjects pre and post LDL apheresis (n=7 per group) to reduce the cholesterol content of apoE macrophages.

FIG. 8A-8D show that 2-HOBA does not impact body weight, water intake, food consumption or lipoprotein profile in hypercholesterolemic Ldlr^{-/-} mice. The body weight (FIG. 8A), water intake (FIG. 8B), and diet consumption (FIG. 8C) were measured in the Ldlr^{-/-} mice consuming a Western diet for 16 weeks and treated with 1 g/L 2-HOBA, 4-HOBA, or vehicle. (FIG. 8D) The plasma was pooled from hypercholesterolemic Ldlr^{-/-} mice (4 mice/group) that were fasted for 6 hours. Fast performance liquid chromatography (FPLC) was performed using a Superose 6 column. Total cholesterol was measured by an enzymatic assay and the average is shown for two pooled plasma samples per group of mice.

FIG. 9A-9E show that 2-HOBA reduces atherosclerotic lesions in the hypercholesterolemic male Ldlr^{-/-} mice. 12-week old male Ldlr^{-/-} mice were pretreated with 1 g/L 2-HOBA or vehicle (water) for 2 weeks and then the treatment was continued for 16 weeks during which the mice were fed a Western diet. (FIG. 9A) Representative images show Oil-Red-O stain in the proximal aorta root sections. (FIG. 9B) Quantitation of the mean Oil-Red-O stainable lesion area in aortic root sections. (FIG. 9C) Representative images show Oil-Red-O staining in open-pinned aortas and (FIG. 9D) the quantitation of en face lesion area. (FIG. 9E) 2-HOBA does not affect the cholesterol levels of male Ldlr^{-/-} mice. (B, D, and E) N=9 or 10 per group, **p<0.05, *** p<0.001, Student t test.

FIGS. 10A and 10B show that 2-HOBA does not impact anti-MDA antibody interaction with MDA-BSA. A series of doses of MDA-BSA or MDA alone were incubated with 1× or 5×2-HOBA. Then 2 μl of each sample was loaded onto HyBond-C membrane, and incubated with the blocking buffer, primary anti-MDA antibody and fluorescent secondary antibody after vigorous washing. The image was captured by the Odyssey system (FIG. 10A) and quantitated by ImageJ software (FIG. 10B).

FIG. 11A-11C show the concentration of 2-HOBA or 4-HOBA was measured in plasma and tissues from Ldlr^{-/-} mice. A. Eight week old male Ldlr^{-/-} mice were fed WD for 16 weeks and were continuously treated with water containing either 2-HOBA (n=9) or 4-HOBA (n=6). Plasma was collected 30 min after oral gavage of mice with either 2-HOBA or 4-HOBA (5 mg each mouse). (Mean±SEM shown for each, p=0.388 Mann-Whitney Test.) B-C. Levels of HOBA were measured in the aorta and heart of male Ldlr^{-/-} mice consuming a chow diet 30 min after oral gavage of 2-HOBA or 4-HOBA (5 mg each mouse). (Mean±SEM shown for each, N.S. Student t test). The levels of 2-HOBA or 4-HOBA were measured in the plasma and tissues using LC/MS as described in the Methods.

FIG. 12A-12D show the levels of 2-HOBA or 4-HOBA in plasma and tissues from C57BL/6J mice. A. Plasma samples were collected from female C57BL/6J mice on a chow diet after intraperitoneal injection of 1 mg 2-HOBA (n=3) or 1 mg 4-HOBA (n=3). *p<0.01, Student t test. B-D. Levels of 2-HOBA and 4-HOBA in liver (B), spleen (C), and kidney (D) of WT mice 30 min after intraperitoneal injection. (Mean±SEM shown for each, N.S. Student t test). The levels of 2-HOBA or 4-HOBA were measured in the plasma and tissues using LC/MS as described in the Methods.

FIG. 13 shows detection of metabolites of isolevuglandin modified 2-HOBA (IsoLG-2-HOBA) in liver of 2-HOBA treated Ldlr^{-/-} mice. Putative metabolites were identified as described in supplemental methods. Representative chromatographs for livers from mice treated with 2-HOBA (left) and 4-HOBA (right) are shown for the three most abundant IsoLG-HOBA metabolites (three upper panels) and the internal standard (lower panel). One potential structure of each metabolite is shown on the left of the chromatograph.

FIG. 14A-14D show MDA-2-HOBA adducts versus −4-HOBA adducts were more readily formed in vivo. Urine samples were collected for 16 h after oral gavage of male Ldlr^{-/-} mice on a WD with either 2-HOBA or 4-HOBA. After 16 h, the Ldlr^{-/-} mice were sacrificed and the HOBA-propenal adducts in urine (14A), liver (14B), kidney (14C) and spleen (14D) were measured using LC-MS/MS as described in methods (14A-D) (Mann-Whitney test, ** indicates p<0.01 and *** indicates p<0.001).

FIG. 15 shows 2-HOBA does not impact urine F2-IsoP in hypercholesterolemic Ldlr^{-/-} mice. The urine F2-IsoP levels were measured by LC/MS/MS) from Ldlr^{-/-} mice consuming a western diet for 16 weeks and treated with 1 g/L 2-HOBA, 4-HOBA, or vehicle. N=5 or 6 per group, p=0.43, Kruskal-Wallis test. Urinary creatinine levels were measured for normalization.

FIG. 16 shows levels of cytokines in serum of Ldlr^{-/-} fed a chow diet for 6 weeks and continuously treated with water alone or containing 1 g/L of either 2-HOBA or 4-HOBA. Serum IL-1β, IL-6 and TNF-α levels were measured by ELISA (R&D System). N=7 or 8 mice per group, N.S., One-way ANOVA with Bonferroni's post hoc test.

FIG. 17 shows WT macrophages were treated with or without 100 μM H₂O₂ with or without increasing concentrations of 2-HOBA for 24 hours. Total RNA was isolated and purified, cDNA was synthesized, and the mRNA levels of IL-1β, IL-6 and TNF-α were measured by real-time PCR. The data are from three independent experiments. *p<0.05, **p<0.01, ***p<0.001, One-way ANOVA (Bonferroni's post hoc test).

FIG. 18A-18D show that treatment of macrophages with 2-HOBA results in formation of 2-HOBA-MDA adducts. Peritoneal macrophages were isolated from C57BL/6J mice and incubated with 50 μg/mL ox-LDL, and treated with either 250 μm 2-HOBA or 4-HOBA (18A), or 5 μm of 2-HOBA or 4-HOBA (18B, 18C, 18D) for 24 h. Cell samples were collected and the HOBA-MDA adducts were measured using LC-MS/MS as described in supplemental methods. * p<0.05, One-way ANOVA with Bonferroni's post hoc test.

FIG. 19A-19B show that 2-HOBA does not influence Akt signaling in macrophages. WT macrophages were treated with or without vehicle (water), 250 μM 4-HOBA or 2-HOBA for 1 hour, and then incubated with or without 100 nM insulin as indicated for 15 min. Phospho-Akt (S473) and GAPDH were detected by Western Blotting (19A). The band density was quantitated by ImageJ software (19B). Two independent experiments were performed.

FIG. 20 shows the effect of 2-HOBA on prostaglandin metabolites. The urine samples were collected in metabolic cages with 2 mice per cage after 12 weeks of treatment with 2-HOBA or water. The contents of PGE-M (20A), tetranor PGD-M (20B), 2,3-dinor-6-keto-PGF1 (20C) and 11-dehydro TxB2 (20D) were analyzed by LC/MS/MS. (20A-20D) Mean±SEM shown for each, N.S., Mann-Whitney Test.

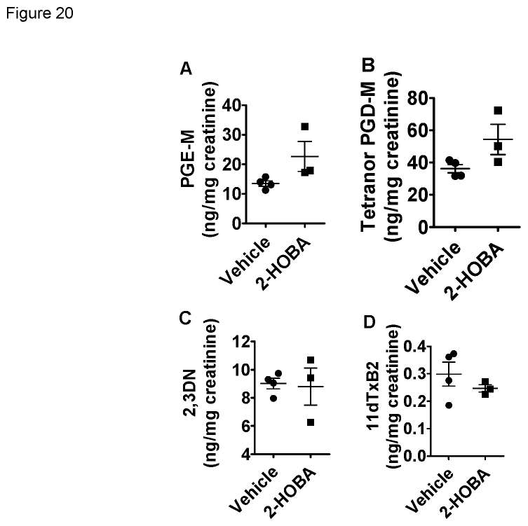

FIG. 21A-D show the effects of 2-HOBA on plasma and LDL MDA adducts in hypercholesterolemic Ldlr^{-/-} mice. (20A) The MDA content in plasma from Ldlr^{-/-} mice consuming a Western diet for 16 weeks and treated with 2-HOBA, 4-HOBA, or vehicle was measured by TBARS Assay(*p<0.05, **p<0.01). (20B) The levels of MDA adducts were measured from LDL isolated from the Ldlr^{-/-} mice by ELISA. N=10 per group, ***p<0.001. (20C) LDL was isolated from control and FH subjects (n=6) pre and post LDL apheresis and the MDA adduct content was measured by ELISA. (20D) LDL was isolated from 2-HOBA, 4-HOBA, or vehicle treated hypercholesterolemic Ldlr^{-/-} mice. WT peritoneal macrophages were incubated for 24 hrs with the LDL and the cellular cholesterol content was measured as described in methods. (20A-D) One-way ANOVA with Bonferroni's post hoc test).

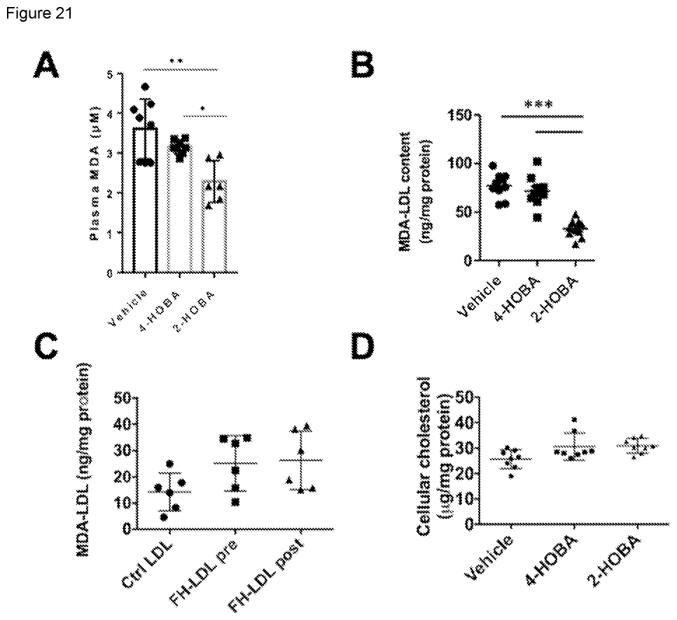

FIG. 22A-B show modification of HDL with increasing concentrations of MDA impaired cholesterol efflux in a dose dependent manner. (22A) The HDL was modified with MDA and the MDA adduct was measured by ELISA. (22B) Apoe^{-/-} peritoneal macrophages were incubated with ac-LDL for 40 h and then incubated for 24 h with 50 ug/mL of HDL or MDA-HDL. The net cholesterol efflux capacity was measured as described in methods (One-way ANOVA with Bonferroni's post hoc test, * indicates p<0.05).

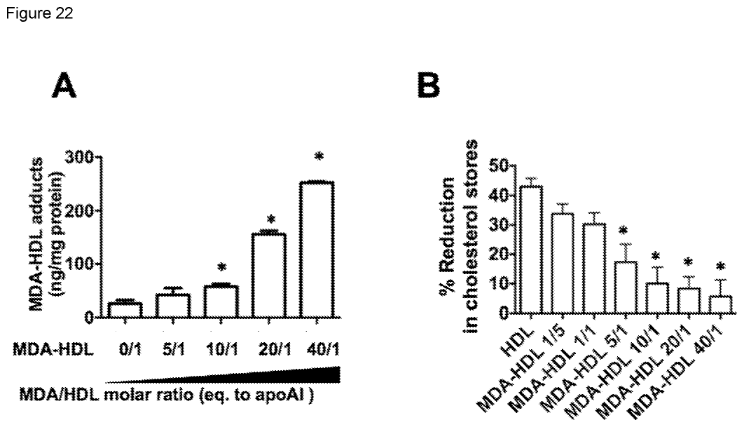

FIG. 23A-B show that the same multiple reaction monitoring (MRM) parameters that detect 2-HOBA aldehyde adducts also detect 4-HOBA aldehyde adducts. Panel A: MRM m/z 259→m/z 107 chromatograph for PITC derivatized 2-HOBA (left) and PITC derivatized 4-HOBA (right). Panel B: MRM chromatograph m/z 472→m/z 107 for IsoLG(hydroxylactam)-2-HOBA (left) and IsoLG(hydroxylactam)-4-HOBA (right). The MRM chromatographs for MDA(propenal)-2-HOBA adduct and MDA(propenal)-4-HOBA adduct have been previously published.

FIG. 24 shows the concentration response curve for the PITC derivative of 4-HOBA differs from that of 2-HOBA when [2H₄]2-HOBA is used as an internal standard and therefore requires use of a correction factor. Varying concentrations (20-400 nmol) of either 2-HOBA or 4-HOBA were mixed with 1 nmol of [₂H₄]2-HOBA, the compounds derivatized with PITC, and then analyzed on LC/MS using either MRM transition m/z 259→m/z 107 or m/z 259→153 for 2-HOBA and 4-HOBA and either m/z 263→m/z 111 or m/z 263→153 for [²H₄]2-HOBA and the measured nmol calculated using the ratio of peak areas. The concentration response slope for each was calculated using GraphPad Prism, and the correction factor for 4-HOBA calculated as the ratio of the two slopes.

### DESCRIPTION OF THE PRESENT INVENTION

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which need to be independently confirmed.

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “optional” or “optionally” means that the subsequently described event or circumstance can or cannot occur, and that the description includes instances where said event or circumstance occurs and instances where it does not.

As used herein, the term “subject” refers to a target of administration. The subject of the herein disclosed methods can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed. As can be seen herein, there is overlap in the definition of treating and preventing.

As used herein, the term “diagnosed” means having been subjected to a physical examination by a person of skill, for example, a physician, and found to have a condition that can be diagnosed or treated by the compounds, compositions, or methods disclosed herein. As used herein, the phrase “identified to be in need of treatment for a disorder,” or the like, refers to selection of a subject based upon need for treatment of the disorder. For example, a subject can be identified as having a need for treatment of a disorder (e.g., a disorder related to inflammation) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “scavenger” or “scavenging” refers to a chemical substance that can be administered in order to remove or inactivate impurities or unwanted reaction products. For example, isolevuglandins irreversibly adduct specifically to lysine residues on proteins. The isolevuglandins scavengers of the present invention react with isolevuglandins before they adduct to the lysine residues. Accordingly, the compounds of the present invention “scavenge” isolevuglandins, thereby preventing them from adducting to proteins.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, optionally substituted alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, optionally substituted alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by a formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA′ where A′ is alkyl or cycloalkyl as defined above. “Alkoxy” also includes polymers of alkoxy groups as just described; that is, an alkoxy can be a polyether such as —OA¹-OA² or —OA¹—(OA²)ₐ-OA³, where “a” is an integer of from 1 to 200 and A¹, A², and A³ are alkyl and/or cycloalkyl groups.

The terms “amine” or “amino” as used herein are represented by a formula NA¹A²A³, where A¹, A², and A³ can be, independently, hydrogen or optionally substituted alkyl, cycloalkyl, alkenyl, cycloalkenyl, alkynyl, cycloalkynyl, aryl, or heteroaryl group as described herein.

The term “hydroxyl” as used herein is represented by a formula —OH.

The term “nitro” as used herein is represented by a formula —NO₂.

The term “pharmaceutically acceptable” describes a material that is not biologically or otherwise undesirable, i.e., without causing an unacceptable level of undesirable biological effects or interacting in a deleterious manner.

Abbreviations used herein include the following: 2-HOBA, 2-hydroxybenzylamine; 4-HOBA, 4-hydroxybenzylamine; MBA, malondialdehyde; 4-HNE, 4-hydroxynonenal; IsoLGs, isolevuglandins; HDL, high-density lipoproteins; LDL, low-density lipoprotein; LDLR, low-density lipoprotein receptor; ApoAI, apolipoprotein AI; ApoB, apolipoprotein B; ROS, reactive oxygen species; IL, interleukin.

In embodiments of the present invention, compounds may be selected from the following formula:

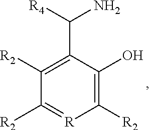

wherein:
R is C—R₂;
each R₂ is independent and chosen from H, substituted or unsubstituted alkyl, halogen, alkyl, substituted or unsubstituted alkoxy, hydroxyl, nitro;
R₄ is H, 2H, substituted or unsubstituted alkyl, carboxyl; and pharmaceutically acceptable salts thereof.

In another embodiment, the compound is selected from the following formula:

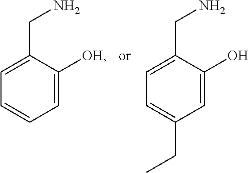

or a pharmaceutically acceptable salt thereof.

In other embodiment, the compound is 2-hydroxybenzylamine, ethyl-2-hydroxybenzylamine, or methyl-2-hydroxybenzylamine.

In another embodiment, the compound is 2-hydroxybenzylamine.

In another embodiment, the compound is selected from the following formula:

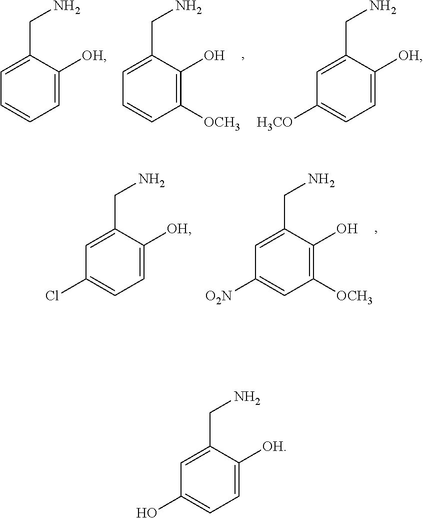

or a pharmaceutically acceptable salt thereof.

In another embodiment, the compound is chosen from:

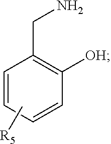

wherein R₅ is H, —CH₃, —CH₂CH₃, —CH(CH₃)—CH₃.

In another embodiment, any of the above compounds is in a pharmaceutical composition comprising said compound and a pharmaceutically acceptable carrier.

In certain aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

As used herein, the term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (-ic and -ous), ferric, ferrous, lithium, magnesium, manganese (-ic and -ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N-thbenzyl ethyl enediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

As used herein, the term “pharmaceutically acceptable non-toxic acids” includes inorganic acids, organic acids, and salts prepared therefrom, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

In practice, the compounds of the invention, or pharmaceutically acceptable salts thereof, of this invention can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds. The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The pharmaceutical compositions of the present invention can comprise a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

The compounds of the present invention can be administered as the sole active pharmaceutical agent, or can be used in combination with one or more other agents useful for treating or preventing various complications, such as, for example, inflammation and other inflammation-related diseases. When administered as a combination, the therapeutic agents can be formulated as separate compositions that are given at the same time or different times, or the therapeutic agents can be given as a single composition.

As indicated herein, the compounds of the present invention may be made up in a solid form (including granules, powders or suppositories) or in a liquid form (e.g., solutions, suspensions, or emulsions). They may be applied in a variety of solutions and may be subjected to conventional pharmaceutical operations such as sterilization and/or may contain conventional adjuvants, such as preservatives, stabilizers, wetting agents, emulsifiers, buffers etc.

Thus, for administration, the compounds of the present invention are ordinarily combined with one or more adjuvants appropriate for the indicated route of administration. For example, they may be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and tableted or encapsulated for conventional administration. Alternatively, they may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

In therapeutic applications, the compounds of the present invention may be administered to a mammalian patient in an amount sufficient to reduce or inhibit the desired indication. Amounts effective for this use depend on factors including, but not limited to, the route of administration, the stage and severity of the indication, the general state of health of the mammal, and the judgment of the prescribing physician. The compounds of the present invention are safe and effective over a wide dosage range. However, it will be understood that the amounts of pyridoxamine actually administered will be determined by a physician, in the light of the above relevant circumstances.

Pharmaceutically acceptable acid addition salts of the compounds suitable for use in methods of the invention include salts derived from nontoxic inorganic acids such as hydrochloric, nitric, phosphoric, sulfuric, hydrobromic, hydriodic, hydrofluoric, phosphorous, and the like, as well as the salts derived from nontoxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, trifluoroacetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacturonate, n-methyl glutamine, etc. (see, e.g., Berge et al., J. Pharmaceutical Science, 66: 1-19 (1977).

The acid addition salts of the basic compounds are prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Also disclosed are methods for treating or inhibiting atherosclerosis in a subject comprising the step of co-administering to the mammal at least one compound in a dosage and amount effective to inhibit platelet activation, the compound having a structure represented by a compound of the following formula:

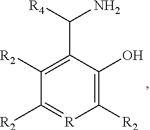

wherein:
R is C—R₂;
each R₂ is independent and chosen from H, substituted or unsubstituted alkyl, halogen, alkyl, substituted or unsubstituted alkoxy, hydroxyl, nitro;
R₄ is H, 2H, substituted or unsubstituted alkyl, carboxyl; and pharmaceutically acceptable salts thereof.
   with a drug having a known side-effect of treating or inhibiting atherosclerosis.

Thus, the disclosed compounds may be used as single agents or in combination with one or more other drugs in the treatment, prevention, control, amelioration or reduction of risk of the aforementioned diseases, disorders and conditions for which compounds of the present invention or the other drugs have utility, where the combination of drugs together are safer or more effective than either drug alone. The other drug(s) may be administered by a route and in an amount commonly used therefore, contemporaneously or sequentially with a disclosed compound. When a disclosed compound is used contemporaneously with one or more other drugs, a pharmaceutical composition in unit dosage form containing such drugs and the compound is preferred. However, the combination therapy can also be administered on overlapping schedules. It is also envisioned that the combination of one or more active ingredients and a disclosed compound can be more efficacious than either as a single agent.

In one aspect, the compounds can be coadministered with anti-atherosclerosis agents.

### EXAMPLES

The following examples discuss embodiments of the present invention.

Results

2-HOBA treatment attenuates atherosclerosis without altering plasma cholesterol levels in Ldlr^{-/-} mice.

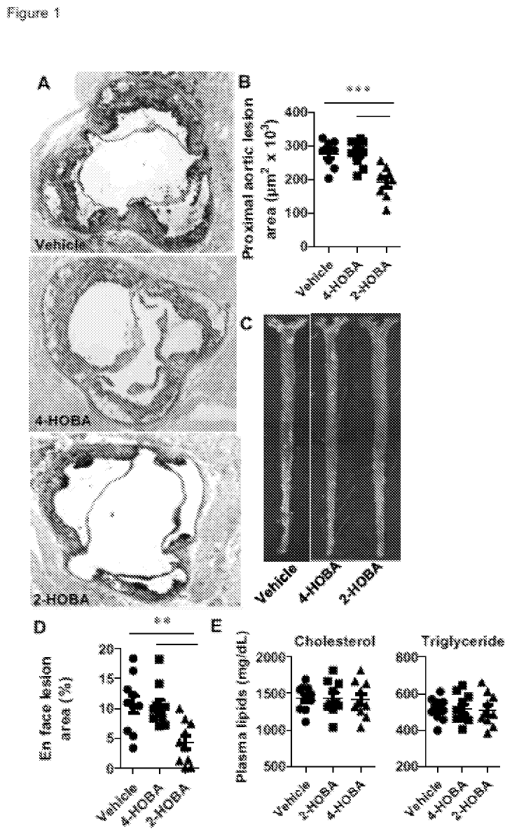

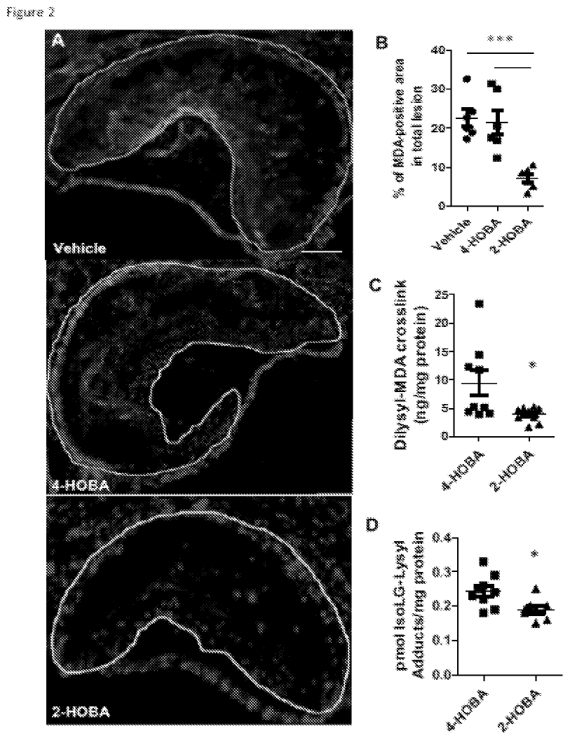

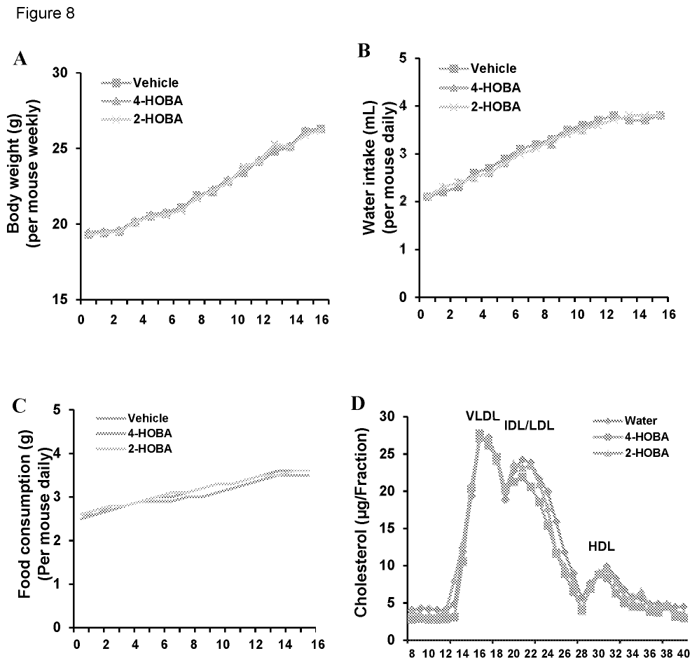

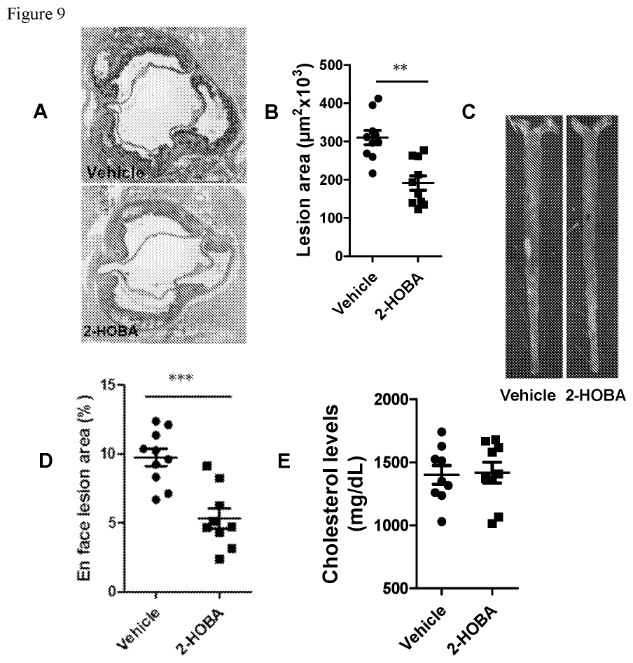

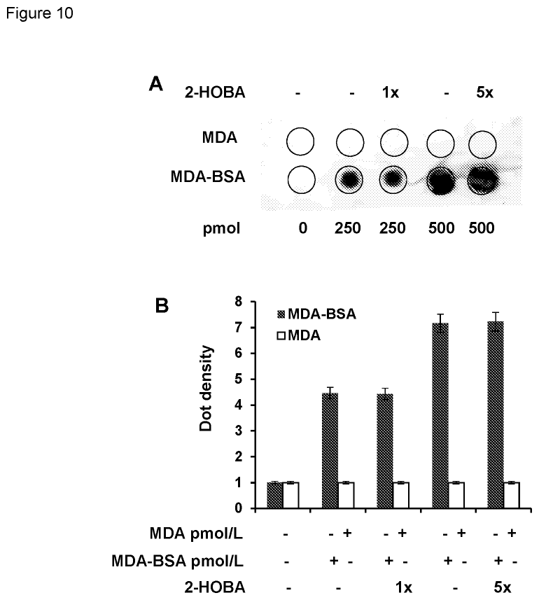

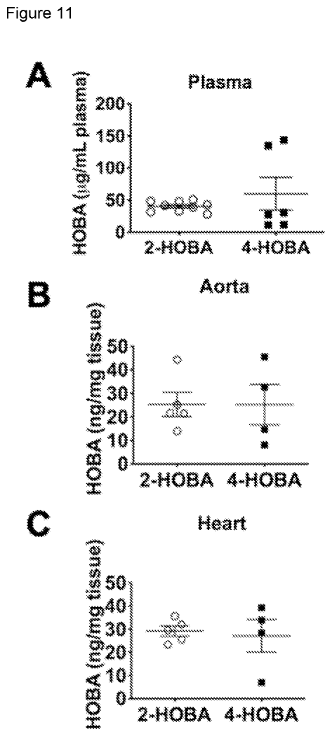

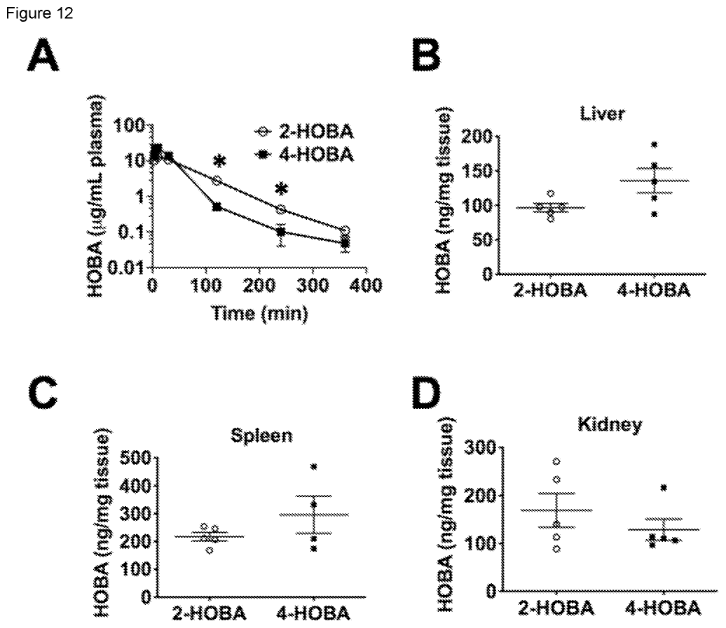

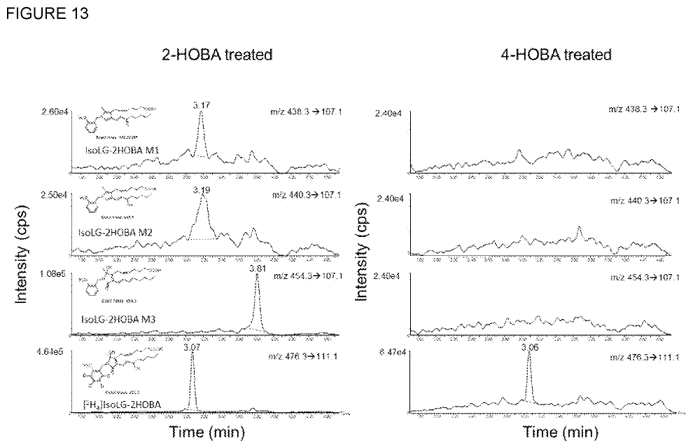

Eight week old, female Ldlr^{-/-} mice were fed a western-type diet for 16 weeks and were continuously treated with vehicle alone (water) or water containing either 2-HOBA or 4-HOBA, an ineffective dicarbonyl scavenger. Treatment with 2-HOBA reduced the extent of proximal aortic atherosclerosis by 31.1% and 31.5%, compared to treatment with either vehicle or 4-HOBA, respectively (FIGS. 1A and 1B). In addition, en face analysis of the aorta demonstrated that treatment of female Ldlr^{-/-} mice with 2-HOBA reduced the extent of aortic atherosclerosis by 60.3% and 59.1% compared to administration of vehicle and 4-HOBA, respectively (FIGS. 1C and 1D). Compared to administration of vehicle or 4-HOBA, 2-HOBA treatment did not affect body weight, water consumption, or diet uptake (FIGS. 8A-8C). In addition, the plasma total cholesterol and triglyceride levels were not significantly different (FIG. 1E), and the lipoprotein distribution was similar between the 3 groups of mice (FIG. 8D). Consistent with these results, treatment of male Ldlr^{-/-} mice with 1 g/L of 2-HOBA, under similar conditions of being fed a western diet for 16 weeks, reduced the extent of proximal aortic and whole aorta atherosclerosis by 37% and 45%, respectively, compared to treatment with water (FIG. 9A-9D) but did not affect the plasma total cholesterol levels (FIG. 9E). A similar reduction in atherosclerosis was observed when male Ldlr^{-/-} mice were treated with 3 g/L of 2-HOBA. Thus, for the first time, the present inventors demonstrated that 2-HOBA treatment significantly decreases atherosclerosis development in an experimental mouse model of FH without changing plasma cholesterol and triglyceride levels. Examination of the proximal aortic MDA adduct content by immunofluorescence staining using an antibody against MDA-protein adducts (Abeam cat#ab6463) shows that the MDA adduct levels were reduced by 68.5% and 66.8% in 2-HOBA treated mice compared to mice treated with vehicle alone or 4-HOBA (FIGS. 2A and 2B). The present inventors determined that the anti-MDA-protein antibody does not recognize either free MDA or MDA-2-HOBA adducts (FIGS. 10A and 10B). In addition, 2-HOBA does not interfere with the antibody recognition of MDA-albumin adducts (FIGS. 10A and 10B). Quantitative measurement of the whole aorta MDA- and IsoLG-lysyl adducts by LC/MS/MS demonstrates that compared to 4-HOBA treatment, administration of 2-HOBA decreased the MDA and IsoLG adduct content by 59% and 23%, respectively (FIGS. 2C and 2D). The present inventors determined by LC/MS/MS that the plasma levels of 2-HOBA in the male Ldlr^{-/-} mice after 16 weeks of treatment with 1 g of 2-HOBA/L of water were 469±38 ng/mL, which is similar to what the present inventors previously reported in C57BL6 mice receiving 1 g/L of 2-HOBA²². In addition, these levels are in the same range as the plasma 2-HOBA levels in humans in a recent safety trial²⁴. The plasma levels of 4-HOBA in the male Ldlr^{-/-} mice after 16 weeks of treatment with 1 g of 4-HOBA/L of water were 25±3 ng/mL. However, the plasma levels of 2-HOBA versus 4-HOBA in male Ldlr^{-/-} mice 30 min after oral gavage of 5 mg were not significantly different (FIG. 11A). In addition, the levels of 2-HOBA and 4-HOBA were similar in the aorta and heart of male Ldlr^{-/-} mice 30 min after oral gavage (FIGS. 11B and 11C). While plasma levels of 4-HOBA after intraperitoneal injection were slightly higher initially than those of 2-HOBA, 4-HOBA appeared to undergo more rapid clearance (FIG. 12A). In addition, the liver, spleen, and kidney levels of 2-HOBA versus 4-HOBA were not significantly different 30 min after intraperitoneal injection (FIGS. 12B-12D). Taken together, the lower levels of 4-HOBA versus 2-HOBA in the male Ldlr^{-/-} mice after 16 weeks of treatment are likely due to differences in clearance as well as in timing of water consumption before sacrifice. Interestingly, the IsoLG-2-HOBA adducts (with masses consistent with potential keto-pyrrole, anhydro-lactam, keto-lactam, pyrrole, and anhydro-hydroxylactam adducts) were present in the hearts and livers of Ldlr^{-/-} mice after 16 weeks on a western diet, whereas IsoLG-4-HOBA adducts were nondetectable (FIG. 13 and the Table, below).

TABLE 1
Table 1: Levels of IsoLG-HOBA metabolites in liver and hearts
of Ldlr^{-/-} fed a Western diet for 16 weeks and continuously
treated with water containing either 2-HOBA or 4-HOBA. Structures
for metabolites 1-3 (M1, M2, M3) are shown in FIG. 13. No
signal for IsoLG-HOBA metabolites were detected in mice treated
with 4-HOBA. Livers and hearts from five mice for each group
were analyzed (Mean ± SEM shown).

| | | |
|---|---|---|
| | 2-HOBA | 4-HOBA |
| analytes | treated | treated |

Liver (nmol/kg)

| | | |
|---|---|---|
| IsoLG-HOBA-M1 | 0.88 ± 0.14 | ND |
| IsoLG-HOBA-M2 | 0.82 ± 0.12 | ND |
| IsoLG-HOBA-M3 | 4.17 ± 1.85 | ND |

Heart (nmol/kg)

| | | |
|---|---|---|
| IsoLG-HOBA-M1 | 0.74 ± 0.21 | ND |
| IsoLG-HOBA-M2 | 0.57 ± 0.35 | ND |
| IsoLG-HOBA-M3 | 0.24 ± 0.17 | ND |

ND = not detected

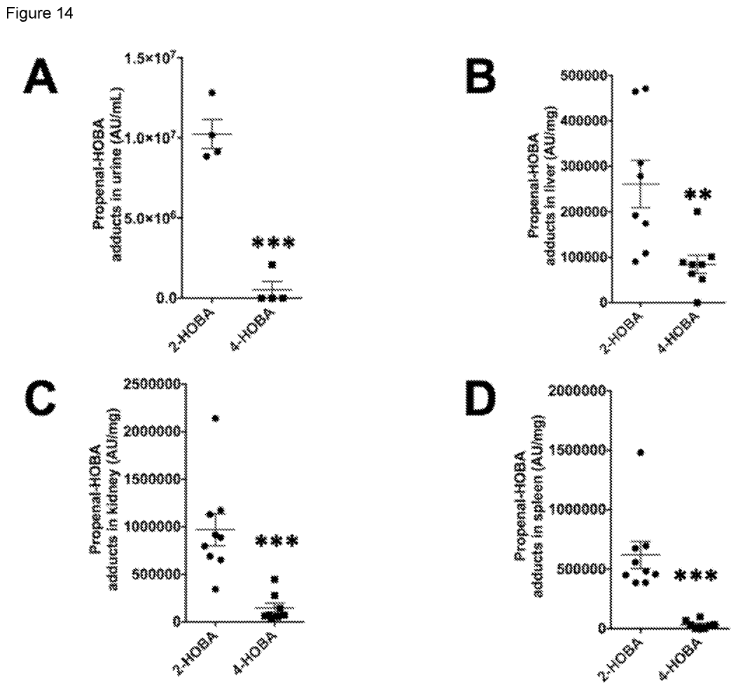

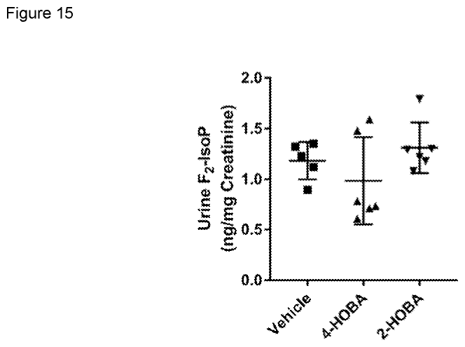

Importantly, the MDA-2-HOBA versus MDA-4-HOBA adducts (with mass consistent with propenal-HOBA adducts) were increased by 19-fold in the urine collected during 16 h after oral gavage (5 mg) treatment of Ldlr^{-/-} mice fed a western diet for 16 weeks (FIG. 14A). In addition, the liver, kidney, and spleen from 2-HOBA versus 4-HOBA treated Ldlr^{-/-} mice also contained 3-, 5-, and 11-fold more propenal-HOBA adducts 16 h post oral gavage (FIGS. 14B-14D). Urine F2-isoprostane (IsoP) levels are a measure of systemic lipid peroxidation, and treatment of Apoe^{-/-} mice with the antioxidant alpha tocopherol reduces atherosclerosis and urine F2-IsoP levels^{25, 26}. The present inventors found that the urine F2-IsoP levels were not different in Ldlr^{-/-} mice treated with vehicle, 4-HOBA, and 2-HOBA (FIG. 15), indicating that the effects of 2-HOBA on atherosclerosis were not due to general inhibition of lipid peroxidation or metal ion chelation. Taken together these results support the hypothesis that the impact of 2-HOBA on atherosclerosis is due to reactive lipid dicarbonyl scavenging.

2-HOBA treatment promotes formation of characteristics of more stable atherosclerotic plaques in hypercholesterolemic Ldlr^{-/-} mice.

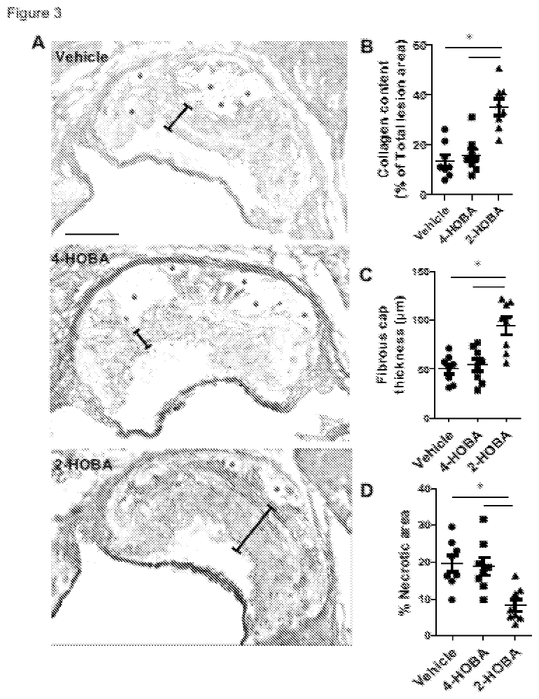

As vulnerable plaques exhibit higher risk for acute cardiovascular events in humans 1, the present inventors examined the effects of 2-HOBA treatment on characteristics of plaque stabilization by quantitating the atherosclerotic lesion collagen content, fibrous cap thickness, and necrotic cores (FIGS. 3A-3D). Compared to administration of vehicle or 4-HOBA, 2-HOBA treatment increased the collagen content of the proximal aorta by 2.7- and 2.6-fold respectively (FIGS. 3A and 3B). In addition, the fibrous cap thickness was 2.31- and 2.29-fold greater in lesions of 2-HOBA treated mice versus vehicle and 4-HOBA treated mice (FIGS. 3A and 3C). Importantly, the % of necrotic area in the proximal aorta was decreased by 74.8% and 73.5% in mice treated with 2-HOBA versus vehicle and 4-HOBA (FIGS. 3A and 3D). Taken together, these data show that 2-HOBA suppresses the characteristics of vulnerable plaque formation in the hypercholesterolemic Ldlr^{-/-} mice.

2-HOBA treatment promotes cell survival and efferocytosis and reduces inflammation.

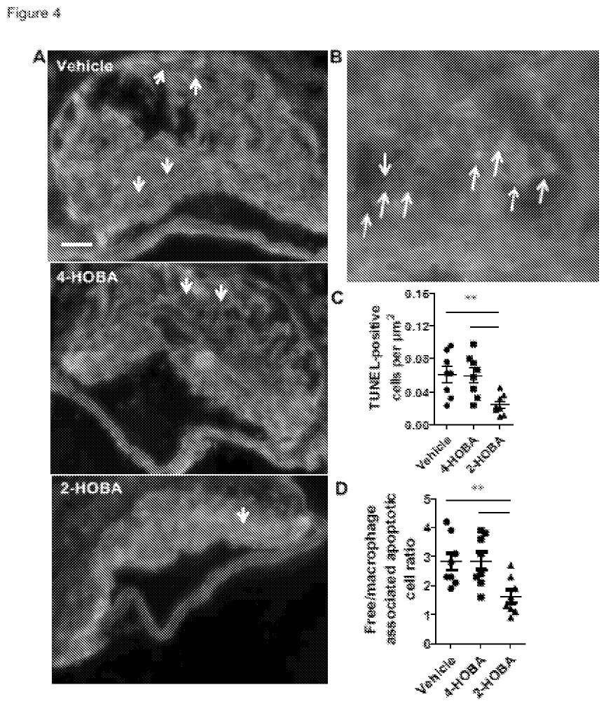

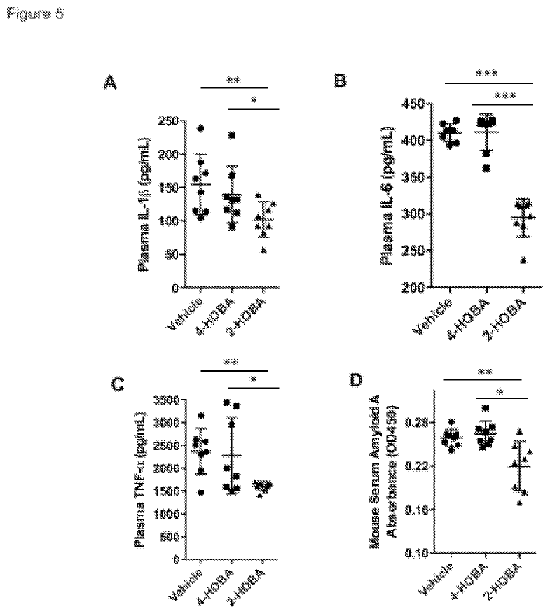

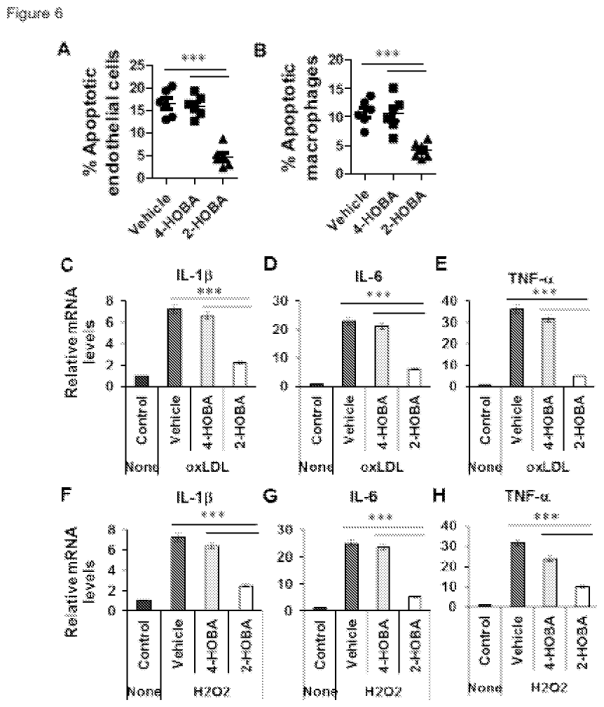

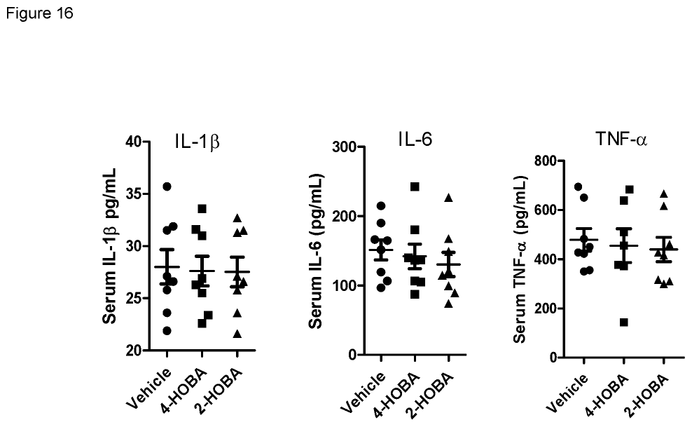

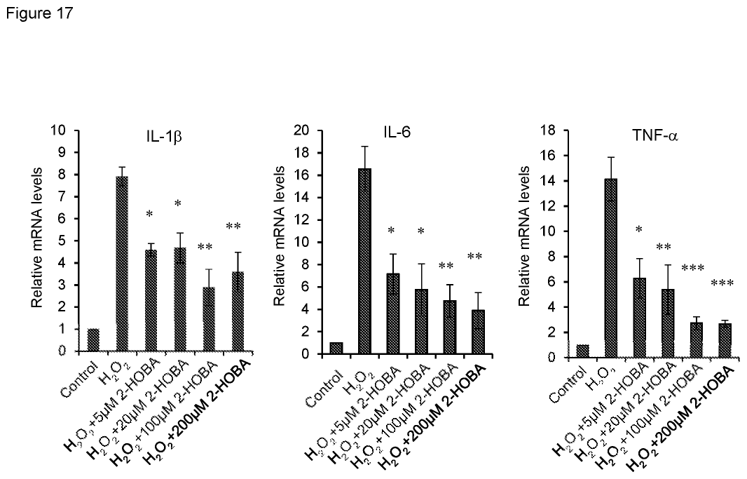

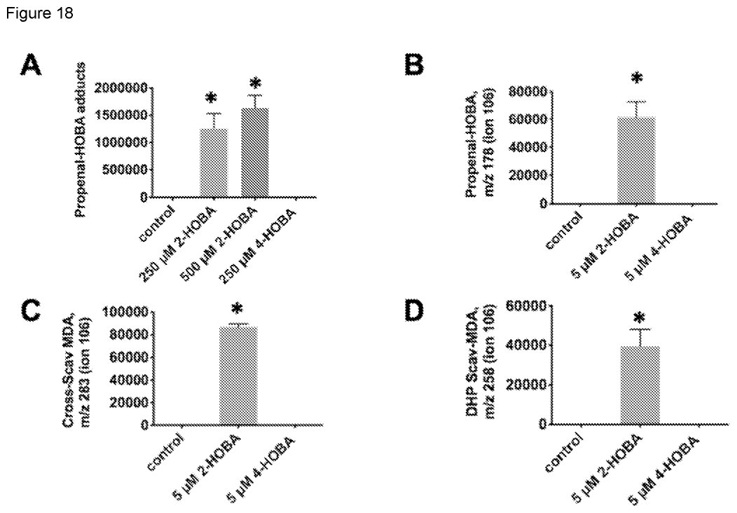

As enhanced cell death and insufficient efferocytosis promote necrotic core formation and destabilization of atherosclerotic plaques, the present inventors next examined the effects of 2-HOBA treatment on cell death and efferocytosis in atherosclerotic lesions in the proximal aorta (FIGS. 4A-4D). Compared to treatment with either vehicle or 4-HOBA, the number of TUNEL positive cells was reduced by 72.9% and 72.4% in the proximal aortic lesions of 2-HOBA treated mice (FIGS. 4A and 4C). The present inventors also examined the impact of 2-HOBA on efferocytosis in the atherosclerotic lesions, and the number of TUNEL positive cells not associated with macrophages was increased by 1.9- and 2.0-fold in lesions of mice treated with vehicle and 4-HOBA versus 2-HOBA (FIGS. 4B and 4D), supporting the ability of reactive lipid dicarbonyl scavenging to maintain efficient efferocytosis. Consistent with lesion necrosis being linked to enhanced inflammation, the serum levels of IL-1β, IL-6, TNF-α, and serum amyloid A were reduced in 2-HOBA versus 4-HOBA or vehicle treated Ldlr^{-/-} mice (FIG. 5), suggesting that reactive dicarbonyl scavenging decreased systemic inflammation. In contrast to results in Ldlr^{-/-} mice fed a western diet, Ldlr^{-/-} mice consuming a chow diet had lower plasma levels of IL-1β, IL-6, and TNF-α, and 2-HOBA treatment had no impact on cytokine levels in the chow fed mice (FIG. 16). These results support the ability of a high-fat western diet to induce oxidative stress and inflammation in Ldlr^{-/-} mice. As studies have demonstrated that incubation of cells with either H₂O₂^{15, 25, 26} or oxidized LDL^{27, 28, 29} induces lipid peroxidation, inflammation, and death, the present inventors next determined the in vitro effects of reactive dicarbonyl scavenging on the cellular response to oxidative stress. Examination of the susceptibility of macrophages and endothelial cells to apoptosis in response to H₂O₂ treatment demonstrates that compared to incubation with vehicle or 4-HOBA, 2-HOBA markedly decreased the number of apoptotic cells in both macrophage and endothelial cell cultures (FIGS. 6A and 6B). In addition, 2-HOBA treatment significantly reduced the macrophage inflammatory response to oxidized LDL as shown by the decreased mRNA levels of IL-1β, IL-6, and TNF-α. (FIGS. 6C-6E). Similar results were observed for the impact of 2-HOBA on the inflammatory cytokine response of macrophages treated with H₂O₂ versus vehicle or 4-HOBA treatment (FIGS. 6F-6H). In addition, the levels of IL-1β, IL-6, and TNF-α mRNA were significantly reduced in macrophages treated with only 5 μM 2-HOBA (615 ng/mL) in the presence of H₂O₂ (FIG. 17). Consistent with the 2-HOBA effects on cell death and inflammation being due to scavenging reactive dicarbonyls, the levels of MDA-2-HOBA (propenal-2-HOBA) versus MDA-4-HOBA adducts were increased in cells treated with oxidized LDL and up to 500 μM 2-HOBA (FIG. 18A). Even in cells incubated just 5 μM 2-HOBA, significant levels of propenal-2-HOBA were formed as well as DHP-MDA-2-HOBA, and crosslinked MDA-2-HOBA adducts were detected (FIGS. 18B-D). In addition, 2-HOBA did not have a direct effect on prosurvival, anti-inflammatory signaling in the absence of oxidative stress,³⁰ as there was no difference in pAKT levels in macrophages treated with vehicle, 4-HOBA, and 2-HOBA in the absence and presence of insulin (FIG. 16). Due to the striking impact on inflammatory cytokines in vivo, the present inventors also measured urinary prostaglandins to evaluate whether 2-HOBA might be inhibiting cyclooxygenase (COX). Urine samples were analyzed for 2,3-dinor-6-keto-PGF1, 11-dehydro TxB2, PGE-M, PGD-M by LC/MS. The present inventors found that there were no significant differences in levels of these major urinary prostaglandin metabolites of Ldlr^{-/-} mice treated with 2-HOBA compared to the vehicle control (FIG. 17), indicating that 2-HOBA was not significantly inhibiting COX in vivo in mice. Taken together, these data show that 2-HOBA treatment maintains efficient efferocytosis in vivo and prevents apoptosis and inflammation in response to oxidative stress by scavenging reactive dicarbonyls.

Effects of 2-HOBA on MDA modification and function of lipoproteins and the impact of familial hypercholesterolemia on lipoprotein MDA adduct content and function.

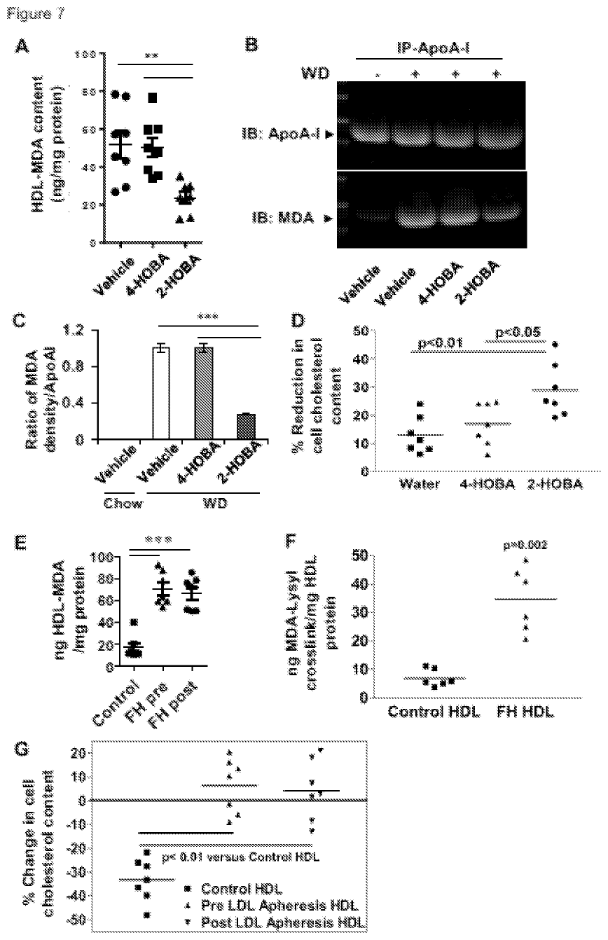

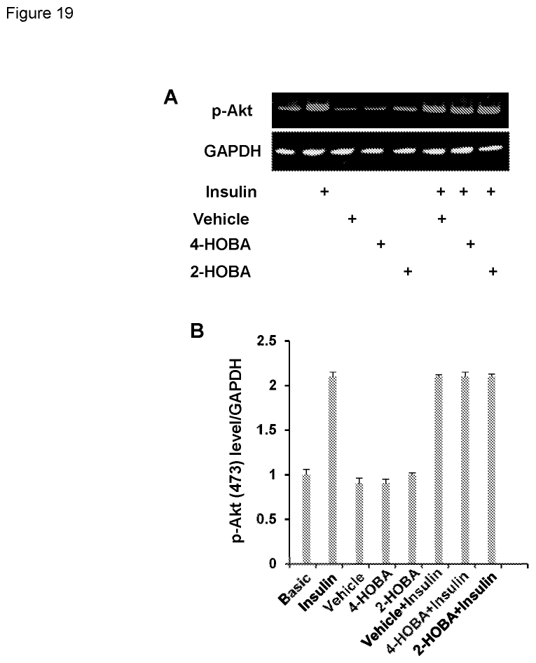

Treatment of the Ldlr^{-/-} mice fed a western diet for 16 weeks with 2-HOBA versus 4-HOBA or vehicle decreased the plasma levels of MDA (FIG. 18A). Compared to treatment with either vehicle or 4-HOBA, the MDA adduct content in isolated LDL measured by ELISA was reduced by 57% and 54%, respectively, in Ldlr^{-/-} mice treated with 2-HOBA (FIG. 18B). By comparison, LDL from control and FH subjects contained similar amounts of MDA adducts, which were not significantly different (FIG. 18C). MDA modification of LDL induces foam cell formation and examination of the ability of LDL from 2-HOBA versus 4-HOBA or vehicle treated Ldlr^{-/-} mice to enrich cells with cholesterol was not different (FIG. 18D). Similar results were observed with FH versus control LDL. This observation was due to the plasma LDL from FH subjects and hypercholesterolemic Ldlr^{-/-} mice being insufficiently modified with MDA to induce cholesterol loading as the present inventors determined by in vitro modification of LDL that the MDA content must be 2500 ng/mg LDL protein to enrich cells with cholesterol. As oxidative modification of HDL impairs its functions, the present inventors next examined the effects of 2-HOBA treatment on HDL MDA content and function. Treatment of Ldlr^{-/-} mice with 2-HOBA reduced the MDA adduct content of isolated HDL as measured by ELISA by 57% and 56% (FIG. 7A) compared to treatment with either vehicle or 4-HOBA. Next, the present inventors examined the effects of 2-HOBA on apoAl MDA adduct formation. ApoAI was isolated from plasma by immunoprecipitation, and MDA-apoAl was detected by western blotting with the antibody to MDA-protein adducts. After 16 weeks on the western-type diet, Ldlr^{-/-} mice treated with vehicle or 4-HOBA had markedly increased plasma levels of MDA-apoAl compared to Ldlr^{-/-} mice consuming a chow diet (FIGS. 7B and 7C). In contrast, treatment of Ldlr^{-/-} mice consuming a western diet with 2-HOBA dramatically reduced plasma MDA-apoAl adducts (FIGS. 7B and 7C). The levels of apoAl were similar among the 4 groups of mice (FIG. 7B). Importantly, the HDL isolated from 2-HOBA treated Ldlr^{-/-} mice was 2.2- and 1.7-fold more efficient at reducing cholesterol stores in Apoe^{-/-} macrophage foam cells versus vehicle and 4-HOBA treated mice (FIG. 7D). In addition, HDL from human subjects with severe FH pre- and post-LDL apheresis (LA) had 5.9-fold and 5.6-fold more MDA adducts compared to control HDL as measured by ELISA (FIG. 7E). The present inventors also found that the dilysyl-MDA crosslink levels as measured by LC/MS/MS were higher in HDL from FH versus control subjects (FIG. 7F). Importantly, HDL from FH versus control subjects lacked the ability to reduce the cholesterol content of cholesterol-enriched Apoe^{-/-} macrophages (FIG. 7G). While the effects of MDA modification of lipid-free apoAl on cholesterol efflux are established,³¹ studies are controversial regarding the impact of modification of HDL^{32, 33}. Therefore, the present inventors determined the impact of in vitro modification of HDL with MDA on the ability of HDL to reduce the cholesterol content of macrophage foam cells as it relates to the MDA adduct content measured by ELISA (FIGS. 19A and 19B). MDA modification of HDL inhibited the net cholesterol efflux capacity in a dose dependent manner, and importantly the MDA-HDL adduct levels which impacted the cholesterol efflux function were in the same range as MDA adduct levels in HDL from FH subjects and hypercholesterolemic Ldlr^{-/-} mice. Taken together, dicarbonyl scavenging with 2-HOBA prevents macrophage foam cell formation by improving HDL net cholesterol efflux capacity. In addition, embodiments of the present invention suggest that scavenging of reactive lipid dicarbonyls could be a relevant therapeutic approach in humans given that HDL from subjects with homozygous FH contain increased MDA and IsoLG and enhanced foam cell formation.

### DISCUSSION

Oxidative stress-induced lipid peroxidation has been implicated in the development of atherosclerosis. Genetic defects and/or environmental factors cause an imbalance between oxidative stress and the ability of the body to counteract or detoxify the harmful effects of oxidation products^{1, 3, 34}. The large body of experimental evidence implicating an important role of lipid peroxidation in the pathogenesis of atherosclerosis previously had stimulated interest in the potential for antioxidants to prevent atherosclerotic cardiovascular disease. Although a few trials of dietary antioxidants in humans demonstrated reductions in atherosclerosis and cardiovascular events, the majority of large clinical outcomes trials with antioxidants have failed to show any benefit in terms of reduced cardiovascular events. Possible reasons for the failure of these trials to reduce cardiovascular events, include inadequate doses of antioxidants being used in the trials^{1, 16} and the inhibition of normal ROS signaling that may be anti-atherogenic³⁵.

Peroxidation of lipids in tissues/cells or in blood produces a number of reactive lipid carbonyls and dicarbonyls including 4-hydroxynonenal, methylglyoxal, malondialdehyde, 4-oxo-nonenal, and isolevuglandins. These electrophiles can covalently bind to proteins, phospholipids, and DNA causing alterations in lipoprotein and cellular functions^{1, 10, 11}. Treatment with scavengers of reactive lipid carbonyl and dicarbonyl species represents a novel alternative therapeutic strategy that will decrease the adverse effects of a particular class of bioactive lipids without completely inhibiting the normal signaling mediated by ROS³⁵. A number of compounds with the potential to scavenge carbonyls have been identified, with individual compounds preferentially reacting with different classes of carbonyls so that the effectiveness of a scavenging compound in mitigating disease can serve as an indicator that their target class of carbonyl contributes to the disease process³⁵. Previous studies found that scavengers of methylglyoxal and glyoxal, such as aminoguanidine and pyridoxamine, reduce atherosclerotic lesions in streptozotocin-treated Apoe^{-/-} mice^{36, 37}. Similarly, scavengers of α-β-unsaturated carbonyls (e.g. HNE and acrolein) such as carnosine and its derivatives, also reduce atherosclerosis in Apoe^{-/-} mice or streptozotocin-treated Apoe^{-/-} mice^{38, 39, 40}. These previously tested scavenger compounds are poor in vivo scavengers of lipid dicarbonyls such as IsoLG and MDA³⁵. Therefore, the present inventors sought to examine the potential of 2-HOBA, an effective scavenger of IsoLG and MDA, to prevent the development of atherosclerosis in Ldlr^{-/-} mice.

The present inventors have recently reported that 2-HOBA can reduce isolevuglandin-mediated HDL modification and dysfunction⁴¹. The present invention is the first to examine the effects of dicarbonyl scavenging on atherosclerosis, and the present inventors demonstrate that compounds of the present invention, including the dicarbonyl scavenger, 2-HOBA, significantly reduces atherosclerosis development in the hypercholesterolemic Ldlr mouse model (FIG. 1). Importantly, embodiments of the invention show that 2-HOBA treatment markedly improves features of the stability of the atherosclerotic plaque as evidenced by decreased necrosis and increased fibrous cap thickness and collagen content (FIG. 3). Consistent with the proinflammatory effects of reactive dicarbonyls⁴¹ and the impact on lesion necrosis, 2-HOBA reduced systemic inflammation by neutralizing reactive dicarbonyls (FIGS. 5 and 6). Furthermore, dicarbonyl scavenging reduced in vivo MDA modification of HDL, consistent with the notion that preventing dicarbonyl modification of HDL improves its net cholesterol efflux capacity (FIG. 7). The present inventors previously showed that IsoLG modification increases in HDL from subjects with familial hypercholesterolemia⁴¹, and this current study shows that MDA modification is similarly increased (FIG. 7), suggesting these modifications contribute to the enhanced foam cell formation induced by FH-HDL (FIG. 7). Taken together, dicarbonyl scavenging using 2-HOBA offers therapeutic potential in reducing atherosclerosis development and the risk of clinical events resulting from formation of vulnerable atherosclerotic plaques.

As embodiments of the invention show that 2-HOBA reduces atherosclerosis development without decreasing plasma cholesterol levels (FIG. 1), without being bound by theory or mechanism, the atheroprotective effects of 2-HOBA are likely due to scavenging bioactive dicarbonyls. Consistent with this concept, the atherosclerotic lesion MDA- and IsoLG-lysyl adducts were decreased in 2-HOBA treated Ldlr^{-/-} mice (FIG. 2). That the effects of 2-HOBA are mediated by their action as dicarbonyl scavengers is further supported by the result that 4-HOBA, a geometric isomer of 2-HOBA, which is not an effective scavenger in vitro, is not atheroprotective and by the finding that MDA- and IsoLG-2-HOBA were abundantly formed versus −4-HOBA adducts (FIGS. 12 and 13 and Table 1) in hypercholesterolemic Ldlr^{-/-} mice. In addition, the levels of urine F₂-isoprostanes were not significantly different between 2-HOBA and 4-HOBA treated Ldlr^{-/-} mice suggesting that the atheroprotective effects are not via inhibition of lipid peroxidation or chelating metal ions (FIG. 15). A possible factor in the comparisons is that 4-HOBA is cleared more rapidly compared to 2-HOBA in vivo, raising the possibility that the finding that 4-HOBA dicarbonyl adducts were very low to undetectable in vivo could in part be due to the lower concentrations of 4-HOBA in tissues. While initial plasma concentrations after oral or intraperitoneal distribution do not significantly differ, elimination of 4-HOBA from the plasma compartment occurs more rapidly than for 2-HOBA. These differences in clearance raise the possibility that our finding that 4-HOBA dicarbonyl adducts were very low to undetectable in vivo could be due in part to the lower concentrations of 4-HOBA in tissues. However, it is important to note that the liver, spleen, and kidney contained similar levels of 2-HOBA versus 4-HOBA 30 min after intraperitoneal dosing (FIG. 12). Furthermore, the aorta and heart levels of 2-HOBA and 4-HOBA were similar 30 min after oral gavage of Ldlr^{-/-} mice (FIG. 11) suggesting equal access to scavenge reactive dicarbonyls in the developing atherosclerotic lesion. Previous in vitro studies demonstrated poor reactivity of 4-HOBA versus 2-HOBA with reactive dicarbonyls²¹. Consistent with the lack of reactivity of 4-HOBA with reactive dicarbonyls in biological systems, when macrophages were treated in vitro with ox-LDL in the presence of 2-HOBA or 4-HOBA, 2-HOBA-MDA adducts were readily detected, whereas 4-HOBA-MDA adducts were undetectable (FIG. 18). The concept that 2-HOBA versus 4-HOBA is an efficient in vivo scavenger of reactive dicarbonyls is substantiated by the finding that 19-fold more MDA-2-HOBA adducts accumulated in the urine during 16 h post oral gavage of Ldlr^{-/-} mice (FIG. 14A). The increased levels of MDA-2-HOBA versus MDA-4-HOBA adducts in liver, spleen, and kidney 16 h after oral gavage of Ldlr^{-/-} mice also strongly support that 2-HOBA is an effective in vivo dicarbonyl scavenger but 4-HOBA is not. Taken together, the present invention shows that atherosclerosis can be prevented by utilizing 2-HOBA to remove dicarbonyls strengthens the hypothesis that reactive dicarbonyls contribute to the pathogenesis of atherogenesis and raises the therapeutic potential of dicarbonyl scavenging in atherosclerotic cardiovascular disease. In this regard, the present inventors found that Ldlr^{-/-} mice treated with 1 g of 2-HOBA/L of water had plasma levels of 2-HOBA that were similar to humans receiving oral doses of 2-HOBA in recent safety trial in humans²⁴.

HDL mediates a number of atheroprotective functions and evidence has mounted that markers of HDL dysfunction, such as impaired cholesterol efflux capacity, may be a better indicator of CAD risk than HDL-C levels^{1, 7, 42, 43, 44}. Patients with FH have previously been shown to have impaired HDL cholesterol efflux capacity, indicative of dysfunctional HDL^{45, 46}. Embodiments of the present invention show that consumption of a western diet by Ldlr^{-/-} mice results in enhanced MDA-apoAl adduct formation (FIG. 7), and that 2-HOBA treatment dramatically reduces modification of both apoAl and HDL with MDA. Similarly, FH patients had increased plasma levels of MDA-HDL adducts. In addition, in vitro modification of HDL with MDA resulted in decreased net cholesterol efflux capacity, similar to what the present inventors showed previously with IsoLG⁴¹, and these effects which were observed with HDL containing MDA adducts in the same range as FH subjects and hypercholesterolemic mice (FIG. 7 and FIG. 19). Results do not agree with other studies showing that MDA modification of HDL does not significantly impact cholesterol efflux from cholesterol-enriched P388D₁ macrophages, which may be due to differences in modification conditions or cell type³². Findings are consistent with studies by Shao and colleagues demonstrating that modification of lipid-free apoAl with MDA blocks ABCA1 mediated cholesterol efflux³¹. In addition, studies have shown that long term cigarette smoking causes increased MDA-HDL adduct formation, and smoking cessation leads to improved HDL function with increased cholesterol efflux capacity⁴⁷. In line with these results, the present inventors found that HDL isolated from 2-HOBA versus vehicle and 4-HOBA treated mice has enhanced capacity to reduce cholesterol stores in macrophage foam cells (FIG. 7). Furthermore, HDL from human subjects with FH had markedly increased MDA adducts and severely impaired ability to reduce macrophage cholesterol stores pre- and post-LDL apheresis (FIG. 7). Thus, one of the atheroprotective mechanisms of 2-HOBA is likely through preventing formation of dicarbonyl adducts of HDL proteins, thereby preserving HDL net cholesterol efflux function. In addition to decreasing HDL oxidative modification, embodiments of the present invention show that 2-HOBA treatment decreases the in vivo MDA modification of plasma LDL. Studies have shown that MDA modification of LDL promotes uptake via scavenger receptors resulting in foam cell formation and an inflammatory response^{48, 49}. The finding that incubation of macrophages with LDL from both 2-HOBA and 4-HOBA treated mice resulted in a similar cholesterol content is consistent with LDL, which is modified with sufficient amounts of MDA, being rapidly removed via scavenger receptors. However, studies have shown that neutralization of MDA-apoB adducts with antibodies greatly enhances atherosclerosis regression in human apoB100 transgenic Ldlr^{-/-} mice^{50, 51} making it likely that the decreased atherosclerosis with 2-HOBA treatment is also due in part to decreased dicarbonyl modification of apoB within the atherosclerotic lesion.

Evidence has mounted that increased oxidative stress in arterial intima cells is pivotal in inducing ER stress, inflammation, and cell death in atherogenesis^{52, 53}. In particular, efficient efferocytosis and limited cell death are critical to preventing the necrosis and excessive inflammation characteristic of the vulnerable plaque^{1, 52, 54}. Embodiments of the present invention demonstrate that treatment with 2-HOBA promotes characteristics of more stable atherosclerotic plaques in Ldlr^{-/-} mice (FIG. 3). Further embodiments show that 2-HOBA treatment decreased the atherosclerotic lesion MDA and IsoLG adduct content (FIG. 2), supporting the ability of dicarbonyl scavenging in the arterial intima to limit oxidative stress induced inflammation, cell death, and destabilization of the plaque. Embodiments of the present invention show that scavenging of dicarbonyls with 2-HOBA in vitro limits oxidative stress induced apoptosis in both endothelial cells and macrophages (FIG. 6). The decreased cell death is likely due in part to the greatly diminished inflammatory response to oxidative stress from dicarbonyl scavenging with 2-HOBA, as evidenced by the dramatic reductions in serum inflammatory cytokines including IL-1β (FIG. 5). These results are particularly relevant given the recent results of the CANTOS trial showing that reducing inflammation with canakinumab, an IL-1β neutralizing monoclonal antibody, can reduce cardiovascular event rates in humans with prior MI and elevated hsCRP⁵⁵. Importantly, treatment with 2-HOBA did not impact levels of urinary prostaglandin metabolites of prostacyclin, thromboxane, PGE2 and PGD2, indicating that 2-HOBA does not result in significant inhibition of cyclooxygenase in mice in vivo (FIG. 17). Furthermore, 2-HOBA treatment maintained efficient efferocytosis and reduced the number of dead cells in the atherosclerotic lesions (FIG. 4). As a result, dicarbonyl scavenging with 2-HOBA promoted features of stable plaques with decreased necrosis and enhanced collagen content and fibrous cap thickness (FIG. 3). Hence, the ability of 2-HOBA to limit death and inflammation in arterial cells in response to oxidative stress and to promote efficient efferocytosis in the artery wall provides a novel atheroprotective mechanism whereby dicarbonyl scavenging promotes features of plaque stabilization and reduces atherosclerotic lesion formation. Results are substantiated by recent studies demonstrating that Ldlr^{-/-} mice expressing the single-chain variable fragment of E06 antibody to oxidized phospholipid have decreased atherosclerosis with stable plaque features including decreased necrosis and systemic inflammation⁵⁶, effects that are likely due in part to neutralization of esterified reactive dicarbonyls. Given the findings that significant residual inflammatory risk for CAD clinical events in humans independent of cholesterol lowering^{55, 57}, these studies highlight reactive dicarbonyls as a target to decrease this risk. The prevention of atherosclerotic lesion formation is clearly an important strategy for the prevention of cardiovascular events.

In conclusion, 2-HOBA treatment suppresses atherosclerosis development in hypercholesterolemic Ldlr^{-/-} mice. The atheroprotective effects of 2-HOBA likely result from preventing dicarbonyl adduct formation with plasma apoproteins and intimal cellular components. Treatment with 2-HOBA decreased the formation of MDA-apoAl adducts thereby maintaining efficient HDL function. In addition, the prevention of MDA-apoB adducts decreases foam cell formation and inflammation. Finally, within the atherosclerotic lesion, dicarbonyl scavenging limited cell death, inflammation, and necrosis thereby effectively promoting characteristics of stable atherosclerotic plaques. As the atheroprotective effect of 2-HOBA treatment is independent of any action on serum cholesterol levels, 2-HOBA offers real therapeutic potential for decreasing the residual CAD risk that persists in patients treated with HMG-CoA reductase inhibitors.

Materials and Methods

Mice

Ldlr^{-/-} and WT on C57BL/6 background mice were obtained from the Jackson Laboratory. Animal protocols were performed according to the regulations of Vanderbilt University's Institutional Animal Care and Usage Committee. Mice were maintained on chow or a Western-type diet containing 21% milk fat and 0.15% cholesterol (Teklad). Eight week old, female Ldlr^{-/-} mice on a chow diet were pretreated with vehicle alone (Water) or containing either 1 g/L of 4-HOBA or 1 g/L of 2-HOBA. 4-HOBA (as hydrochloride salt) was synthesized as previously described²¹. 2-HOBA (as the acetate salt, CAS 1206675-01-5) was manufactured by TSI Co., Ltd. (Missoula, MT) and obtained from Metabolic Technologies, Inc., Ames, IA²⁴. A commercial production lot was used (Lot 16120312), and the purity of the commercial lot was verified to be >99% via HPLC and NMR spectroscopy²⁴. After two weeks, the mice continued to receive these treatments but were switched to a western diet for 16 weeks to induce hypercholesterolemia and atherosclerosis. Similarly, 12 week-old male Ldlr^{-/-} mice were pretreated with vehicle alone (water) or containing 1 g/L of 2-HOBA for two weeks and were then switched to a western diet for 16 weeks to induce hypercholesterolemia and atherosclerosis, while continuing the treatment with 2-HOBA or water alone^{58, 59, 60} Based on the average weight and daily consumption of water per mouse the estimated daily dosage with 1 g/L of 2-HOBA is 200 mg/Kg. The present inventors did not observe differences in mouse mortality among the treatment groups. Eight week old, male Ldlr^{-/-} mice were fed a western diet for 16 weeks and were continuously treated with water containing either 2-HOBA or 4-HOBA. Urine samples were collected using metabolic cages (2 mice in one cage) during 18 h after oral gavage with either 2-HOBA or 4-HOBA (5 mg each mouse).

Cell Culture

Peritoneal macrophages were isolated from mice 72 hours post injection of 3% thioglycollate and maintained in DMEM plus 10% fetal bovine serum (FBS, Gibco) as previously described³⁰. Human aortic endothelial cells (HAECs) were obtained from Lonza and maintained in endothelial cell basal medium-2 plus 1% FBS and essential growth factors (Lonza).

Plasma Lipids and Lipoprotein Distribution Analyses

The mice were fasted for 6 hours, and plasma total cholesterol and triglycerides were measured by enzymatic methods using the reagents from Cliniqa (San-Macros, CA). Fast performance liquid chromatography (FPLC) was performed on an HPLC system model 600 (Waters, Milford, MA) using a Superose 6 column (Pharmacia, Piscataway, NJ).

HDL Isolation from Mouse Plasma and Measurement of HDL Capacity to Reduce Macrophage Cholesterol

HDL was isolated from mouse plasma using HDL Purification Kit (Cell BioLabs, Inc.) following the manufacturer's protocol. Briefly, apoB containing lipoproteins and HDL were sequentially precipitated with dextran sulfate. The HDL was then resuspended and washed. After removing the dextran sulfate, the HDL was dialyzed against PBS. To measure the capacity of the HDL to reduce macrophage cholesterol, Apoe^{-/-} macrophages were cholesterol enriched by incubation for 48 h in DMEM containing 100 μg protein/ml of acetylated LDL. The cells were then washed, and incubated for 24 h in DMEM alone or with 25 μg HDL protein/ml. Cellular cholesterol was measured before and after incubation with HDL using an enzymatic cholesterol assay as described⁶¹.

Human Blood Collection and Measurement of MDA-LDL, MDA-HDL, and MDA-ApoAI

The study was approved by the Vanderbilt University Institutional Review Board (IRB), and all participants gave their written informed consent. The human blood samples from patients with severe FH, who were undergoing LDL apheresis, and healthy controls were obtained using an IRB approved protocol. HDL and LDL were prepared from serum by Lipoprotein Purification Kits (Cell BioLabs, Inc.). Sandwich ELISA was used to measure plasma MDA-LDL and MDA-HDL levels following the manufacturer's instructions (Cell BioLabs, Inc.). Briefly, isolated LDL or HDL samples and MDA-Lipoprotein standards were added onto anti-MDA coated plates, and, after blocking, the samples were incubated with biotinylated anti-apoB or anti-ApoAI primary antibody. The samples were then incubated for 1 h with streptavidin-enzyme conjugate and 15 min with substrate solution. After stopping the reaction, the O.D. was measured at 450 nm wavelength. MDA-ApoAI was detected in mouse plasma by immunoprecipitation of ApoAI and western blotting. Briefly, 50 μl of mouse plasma was prepared with 450 μL of IP Lysis Buffer (Pierce) plus 0.5% protease inhibitor mixture (Sigma), and immunoprecipitated with 10 μg of polyclonal antibody against mouse ApoAI (Novus). Then 25 μL of magnetic beads (Invitrogen) was added, and the mixture was incubated for 1 h at 4° C. with rotation. The magnetic beads were then collected, washed three times, and SDS-PAGE sample buffer with β-mercaptoethanol was added to the beads. After incubation at 70° C. for 5 min, a magnetic field was applied to the Magnetic Separation Rack (New England), and the supernatant was used for detecting mouse ApoAI or MDA. For Western blotting, 30-60 μg of proteins was resolved by NuPAGE Bis-Tris electrophoresis (Invitrogen), and transferred onto nitrocellulose membranes (Amersham Bioscience). Membranes were probed with primary rabbit antibodies specific for ApoAI (Novus NB600-609) or MDA-BSA (Abcam cat #ab6463) and fluorescent tagged IRDye 680 (LI-COR) secondary antibody. Proteins were visualized and quantitated by Odyssey 3.0 Quantification software (LI-COR).

Modification of HDL and LDL with MDA

MDA was prepared immediately before use by rapid acid hydrolysis of maloncarbonyl bis-(dimethylacetal) as described³¹. Briefly, 20 μL of 1 M HCl was added to 200 μL of maloncarbonyl bis-(dimethylacetal), and the mixture was incubated for 45 min at room temperature. The MDA concentration was determined by absorbance at 245 nm, using the coefficient factor 13, 700 M⁻¹ cm⁻¹. HDL (10 mg of protein/mL) and increasing doses of MDA (0, 0.125 mM, 0.25 mM, 0.5 mM, 1 mM) were incubated at 37° C. for 24 h in 50 mM sodium phosphate buffer (pH7.4) containing DTPA 100 μM. Reactions were initiated by adding MDA and stopped by dialysis of samples against PBS at 4° C. LDL (5 mg/mL) was modified in vitro with MDA (10 mM) in the presence of vehicle alone or with 2-HOBA at 37° C. for 24 h in 50 mM sodium phosphate buffer (pH7.4) containing DTPA 100 μM. Reactions were initiated by adding MDA and stopped by dialysis of samples against PBS at 4° C. The LDL samples were incubated for 24 h with macrophages and the cholesterol content of the cells was measured using an enzymatic cholesterol assay as described⁶¹.

Atherosclerosis Analyses and Cross-Section Immunofluorescence Staining

The extent of atherosclerosis was examined both Oil-Red-O-stained cross-sections of the proximal aorta and by en face analysis³⁰. Briefly, cryosections of 10-micron thickness were cut from the region of the proximal aorta starting from the end of the aortic sinus and for 300 μm distally, according to the method of Paigen et al.⁶². The Oil red-O staining of 15 serial sections from the root to ascending aortic region were used to quantify the Oil red-O-positive staining area per mouse. The mean from the 15 serial sections was applied for the aortic root atherosclerotic lesion size per mouse using the KS300 imaging system (Kontron Elektronik GmbH) as described^{63, 64, 65}. All other stains were done using sections that were 40 to 60 μm distal of the aortic sinus. For each mouse, 4 sections were stained and quantitation was done on the entire cross section of all 4 sections. For immunofluorescence staining, 5 μm cross-sections of the proximal aorta were fixed in cold acetone (Sigma), blocked in Background Buster (Innovex), incubated with indicated primary antibodies (MDA and CD68) at 4° C. for overnight. After incubation with fluorescent labeled secondary antibodies at 37 C for 1 hour, the nucleus was counterstained with Hoechst. Images were captured with a fluorescence microscope (Olympus IX81) and SlideBook 6 (Intelligent-Image) software and quantitated using ImageJ software (NIH)⁶⁶.

In vitro Cellular Apoptosis and Analysis of Lesion Apoptosis and Efferocytosis.

Cell apoptosis was induced as indicated and detected by fluorescent labeled Annexin V staining and quantitated by either Flow Cytometry (BD 5 LSRII) or counting Annexin V positive cells in images captured under a fluorescent microscope. The apoptotic cells in atherosclerotic lesions were measured by TUNEL staining of cross-sections of atherosclerotic proximal aortas as previously described³⁰. The TUNEL positive cells not associated with live macrophages were considered free apoptotic cells and macrophage-associated apoptotic cells were considered phagocytosed as a measure of lesion efferocytosis as previously described³⁰.

Masson's Trichrome Staining

Masson's Trichrome Staining was applied for measurement of atherosclerotic lesion collagen content, fibrous cap thickness and necrotic core size following the manufacturer's instructions (Sigma) and as previously described³⁰. Briefly, 5 μm cross-sections of proximal atherosclerotic aorta root were fixed with Bouin's solution, stained with hematoxylin for nuclei (black) and biebrich scarlet and phosphotungstic/phosphomolybdic acid for cytoplasm (red), and aniline blue for collagen (blue). Images were captured and analyzed for collagen content, atherosclerotic cap thickness and necrotic core by ImageJ software as described previously³⁰. The necrotic area is normalized to the total lesion area and is expressed as the % necrotic area.

RNA Isolation and Real-Time RT-PCR

Total RNA was extracted and purified using Aurum Total RNA kit (Bio-Rad) according to the manufacturer's protocol. Complementary DNA was synthesized with iScript reverse transcriptase (Bio-Rad). Relative quantitation of the target mRNA was performed using specific primers, SYBR probe (Bio-Rad), and iTaqDNA polymerase (Bio-Rad) on IQ5 Thermocylcer (Bio-Rad) and normalized with 18S, as described earlier. 18S, IL-1□ and TNF-□ primers used were as described earlier⁶⁷.

Liquid Chromatography-Mass Spectrometry Analysis of Urinary Prostaglandin Metabolites

Concentrations of PGE-M, tetranor PGD-M, 11-dehydro-TxB² (TxB-M) and PGI-M in urine were measured in the Eicosanoid Core Laboratory at Vanderbilt University Medical Center. Urine (1 mL) was acidified to pH 3 with HCl. [²H₄]-2,3-dinor-6-keto-PGF1a (internal standard for PGI-M quantification) and [²H₄]-11-dehydro-TxB₂ were added, and the sample was treated with methyloxime HCl to convert analytes to the O-methyloxime derivative. The derivatized analytes were extracted using a C-18 Sep-Pak (Waters Corp. Milford, MA USA) and eluted with ethyl acetate as previously described⁶⁸. A [²H₆]—O-methyloxime PGE-M deuterated internal standard was then added for PGE-M and PGD-M quantification. The sample was dried under a stream of dry nitrogen at 37° C. and then reconstituted in 75 μL mobile phase A for LC/MS analysis.

LC was performed on a 2.0×50 mm, 1.7 μm particle Acquity BEH C18 column (Waters Corporation, Milford, MA, USA) using a Waters Acquity UPLC. Mobile phase A was 95:4.9:0.1 (v/v/v) 5 mM ammonium acetate:acetonitrile:acetic acid, and mobile phase B was 10.0:89.9:0.1 (v/v/v) 5 mM ammonium acetate:acetonitrile:acetic acid. Samples were separated by a gradient of 85-5% of mobile phase A over 14 min at a flow rate of 375 μl/min prior to delivery to a SCIEX 6500+QTrap mass spectrometer.

Urinary creatinine levels are measured using a test kit from Enzo Life Sciences. The urinary metabolite levels in each sample are normalized using the urinary creatinine level of the sample and expressed in ng/mg creatinine.

Measurement of 2-HOBA and 4-HOBA in Plasma and Tissue

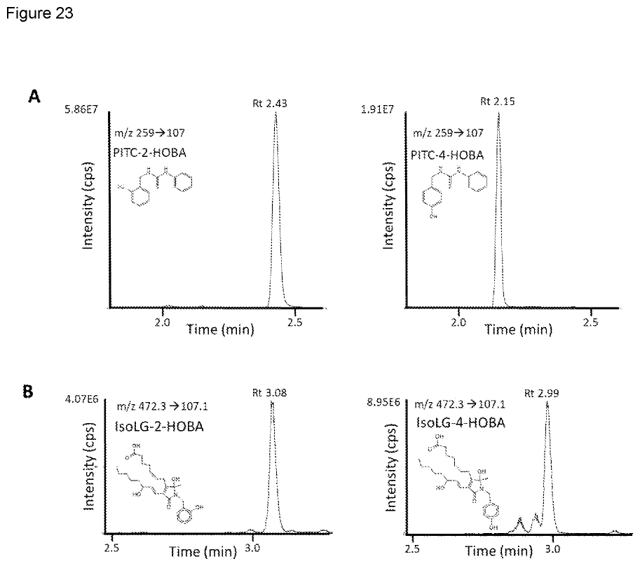

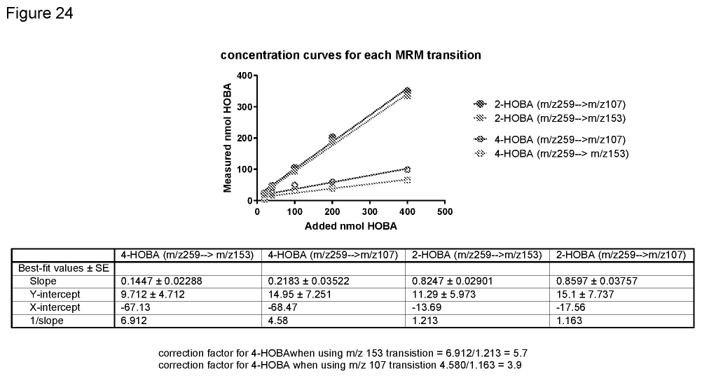

Measurement of 2-HOBA and 4-HOBA was performed by LC/MS after derivatization with phenylisothiocyanate (PITC), and using [²H₄]-2-HOBA as an internal standard as previously described for 2-HOBA⁷¹ (See FIG. 23). For these assays, the Waters Xevo-TQ-Smicro triple quadrupole mass spectrometer operating in positive ion multiple reaction monitoring (MRM) mode monitored the following transitions: for PITC-2-HOBA or PITC-4-HOBA, m/z 259→107@20 eV (quantifier transition) and m/z 259→153 @20 eV (qualifier transition); for PITC-[²H₄]2-HOBA m/z 263 →107@20 eV (quantifier transition), m/z 263→111 @20 eV (qualifier transition). Abundance for PITC-2-HOBA was calculated based on the ratio of peak area versus that of PITC-[²H₄]2-HOBA. Because the transition reactions for PITC-4-HOBA are less efficient than for PITC-2-HOBA, the ratio of peak areas for PITC-4-HOBA/PITC-[^{2l H}₄]2-HOBA was multiplied by the correction factors 3.9 and 5.7 when using the m/z 107 and m/z 153 transition, respectively (See FIG. 24).

Measurement of IsoLG-Lys in Aorta

Isolation and LC/MS measurement of isolevuglandin-lysyl-lactam (IsoLG-Lys) adducts from aorta of 2-HOBA and 4-HOBA treated Ldlr^{-/-} mice were performed using a Waters Xevo-TQ-Smicro triple quadrupole mass spectrometer as previously described⁶⁹.

Detection of IsoLG Adducts of 2-HOBA

To generate an internal standard for quantitation, 10 molar equivalents of the heavy isotope labeled 2-HOBA, [²H₄]2-HOBA, was reacted with synthetic IsoLG⁶⁹ overnight in 1 mM triethylammonium acetate buffer to form IsoLG-2-HOBA adducts, and the adducts separated from unreacted [²H₄]2-HOBA and IsoLG by solid phase extraction (Oasis HLB). The isolated reaction products of IsoLG-2-HOBA were scanned by mass spectrometer (Waters Xevo-TQ-Smicro triple quadrupole MS) operating in limited mass scanning mode to identify major products. Additionally, precursor scanning with the product ion set at m/z 111.1 was used to confirm that the detected products were [²H₄]2-HOBA adducts. Both methods showed that the primary adduct present in the purified IsoLG-[²H₄]2-HOBA internal standard mixture was the IsoLG-[2H4]2-HOBA hydroxylactam adduct, although other adducts including pyrrole, lactam, and the anhydro-species of each of these adducts were also present. Similar species were seen when IsoLG was reacted with non-labeled 2-HOBA and precursor scanning using product ion m/z 107.1 To identify potential 2-HOBA adducts in tissue of treated animals, the present inventors first generated a list of 18 probable IsoLG-HOBA species [pyrrole, lactam, hydroxylactam based on the in vitro reactions of IsoLG and 2-HOBA and then the anhydro-, dinor-, dinor/anhydro-, tetranor-, and keto-(from oxidation of hydroxyl group) metabolites of each of these three adducts based on previous metabolism studies with prostaglandins and isoprostanes]. The present inventors then analyzed liver homogenate from a 2-HOBA treated mouse using LC/MS with the mass spectrometer operating in positive ion precursor scanning mode and the product ion set to m/z 107.1 and collision energy at 20 eV and looked for the presence of any of these precursor ions. Based on these data, the present inventors identified three potential metabolites: M1 precursor ion m/z 438.3, which mass is consistent with either the keto-pyrrole adduct or the anhydro-lactam adduct (both have identical mass). M2 m/z 440.3, which mass is consistent with the pyrrole adduct, and M3 m/z 454.3 which mass is consistent with the anhydro-hydroxylactam adduct or the keto-lactam adduct. When then sought to quantify the amount of the putative IsoLG-HOBA adducts in heart and liver samples as there was not sufficient aorta sample remaining from other analysis available to do this analysis.

For these experiments, liver or heart samples from Ldlr^{-/-} mice treated with 2-HOBA or 4-HOBA were homogenized in 0.5 M Tris buffer solution pH 7.5 containing mixture of antioxidants (pyridoxamine, indomethacin, BHT, TCEP). Total amount of protein in homogenate was determined for normalization. 1 pmol IsoLG-[²H₄]2-HOBA was then added to each homogenate sample as internal standard, the HOBA adducts extracted with ethyl acetate, dried, dissolved in solvent 1 (water with 0.1% acetic acid) and analyzed by LC/MS using Waters Xevo-TQ-Smicro triple quadrupole mass spectrometer operating in positive ion multiple reaction monitoring (MRM) mode, monitoring the following transitions: m/z 438.3→107.1@20 eV for M1; m/z 440→107.1@20 eV for M2; m/z 454→107.1@20 eV for M3; and m/z 476.3→111.1@20 eV for IsoLG-[²H₄]2-HOBA hydroxylactam. Desolvati on temperature: 500° C.; source temperature: 150° C.; capillary voltage: 5 kV, cone voltage: 5 V; cone gas flow 1 L/h; desolvation gas flow 1000 L/h. HPLC condition were as follows: Solvent 1: water with 0.1% acetic acid; Solvent 2, methanol with 0.1% acetic acid; column: Phenomenex Kinetex C8 50×2.1 mm 2.6 u 100 Å, flow rate: 0.4 mL/min; gradient: starting condition 10% B with gradient ramp to 100% B over 3.5 min, hold for 0.5 min, and return to starting conditions over 0.5 min. Abundance for each metabolite was calculated based on the ratio of peak height versus that of internal standard.

Analysis of Dilysyl-MDA Crosslinks by LC/EST/MS/MS

Samples (around 1 mg of protein) were digested with proteases as previously described for lysyl-lactam adducts⁷⁰. Five nanograms of ¹³C₆-dilysyl-MDA crosslink standard were added to each cell sample and dilysyl-MDA crosslinks were purified as previously described⁷¹. The dilysyl-MDA crosslink was quantified by isotopic dilution by LC-EST/MS/MS as previously described⁷¹.

LC/MS/MS quantification of scavenger-MDA adducts.

The scavenger-MDA adducts were extracted, (1) from homogenate of tissue (equivalent of 30 mg) or (2) from cells (1 ml), three times with 500 μl of ethyl acetate. The extract was dried down, resuspended in 100 μl of ACN-water (1:1, v/v with 0.1% formic acid), vortexed, and filtered through a 0.22 μm spin X column. The reactions were analyzed by LC-EST/MS/MS using the column a Phenomenex Kinetex column at a flow rate of 0.1 ml/min. The gradient consisted of Solvent A, water with 0.2% formic acid and solvent B, acetonitrile with 0.2% formic acid. The gradient was as follows: 0-2 min 99.9% A, 2-9 min 99.9-0.1% A, 9-12 min 99.9% B. The mass spectrometer was operated in the positive ion mode, and the spray voltage was maintained at 5,000 V. Nitrogen was used for the sheath gas and auxiliary gas at pressures of 30 and 5 arbitrary units, respectively. The optimized skimmer offset was set at 10, capillary temperature was 300° C., and the tube lens voltage was specific for each compound. SRM of specific transition ions for the precursor ions at m/z 178→107 (propenal-HOBA adduct).

Statistics

Continuous data are summarized as mean±SEM visualized by box plots and bar charts. Between-group differences were assessed with Student's t-test (2 groups) and one-way ANOVA (>2 groups, Bonferroni's correction for multiple comparisons). Their nonparametric counterparts, Mann-Whitney test (2 groups) and nonparametric Kruskal-Wallis test (more than 2 groups, Bunn's correction for multiple comparison) were used when assumptions for parametric methods were not met. The Shapiro-Wilk-Wilk test was used to evaluate normality assumptions. All tests were considered statistically significance at two-sided significance level of 0.05 after correction for multiple comparisons. All statistical analyses were performed in GraphPad PRISM versions 5 or 7.

### REFERENCES

1 Linton M F, Yancey P G, Davies S S, Jerome W G, Linton E F, Song W L, Doran A C, Vickers K C. The Role of Lipids and Lipoproteins in Atherosclerosis. In: De Groot L J, Chrousos G, Dungan K, Feingold K R, Grossman A, Hershman J M, Koch C, Korbonits M, McLachlan R, New M, Purnell J, Rebar R, Singer F, Vinik A, editors. Endotext. South Dartmouth (MA) 2019. http://www.endotext.org/Updated: Jan. 3, 2019. https://www.ncbi.nlm.nih.gov/pubmed/26844337.
2. Sampson U K, Fazio S, Linton M F. Residual cardiovascular risk despite optimal LDL cholesterol reduction with statins: the evidence, etiology, and therapeutic challenges. Curr Atheroscler Rep 14, 1-10 (2012).
3. Anderson T J. Oxidative stress, endothelial function and coronary atherosclerosis. Cardiologia 42, 701-714 (1997).
4. Aviram M. Atherosclerosis: cell biology and lipoproteins—inflammation and oxidative stress in atherogenesis: protective role for paraoxonases. Curr Opin Lipidol 22, 243-244 (2011).
5. Peluso I, Morabito G, Urban L, loannone F, Serafini M. Oxidative stress in atherosclerosis development: the central role of LDL and oxidative burst. Endocr Metab Immune Disord Drug Targets 12, 351-360 (2012).
6. Kontush A, Lindahl M, Lhomme M, Calabresi L, Chapman M J, Davidson W S. Structure of HDL: particle subclasses and molecular components. Handb Exp Pharmacol 224, 3-51 (2015).
7. Riwanto M, Rohrer L, von Eckardstein A, Landmesser U. Dysfunctional HDL: from structure-function-relationships to biomarkers. Handb Exp Pharmacol 224, 337-366 (2015).
8. Linton M F, Tao H, Linton E F, Yancey P G. SR-BI: A Multifunctional Receptor in Cholesterol Homeostasis and Atherosclerosis. Trends Endocrinol Metab, (2017).
9. Brewer H B, Jr., Rader D J. HDL: structure, function and metabolism. Prog Lipid Res 30, 139-144 (1991).
10. Xi H, et al. Potent free radical scavenger, edaravone, suppresses oxidative stress-induced endothelial damage and early atherosclerosis. Atherosclerosis 191, 281-289 (2007).
11. Vasdev S, Gill V D, Singal P K. Modulation of oxidative stress-induced changes in hypertension and atherosclerosis by antioxidants. Exp Clin Cardiol 11, 206-216 (2006).
12. Guo L, et al. Isolevuglandin-type lipid aldehydes induce the inflammatory response of macrophages by modifying phosphatidylethanolamines and activating the receptor for advanced glycation endproducts. Antioxid Redox Signal 22, 1633-1645 (2015).
13. Kirabo A, et al. DC isoketal-modified proteins activate T cells and promote hypertension. J Clin Invest 124, 4642-4656 (2014).
14. Davies S S, et al. Treatment with a gamma-ketoaldehyde scavenger prevents working memory deficits in hApoE4 mice. J Alzheimers Dis 27, 49-59 (2011).
15. Davies S S, et al. Pyridoxamine analogues scavenge lipid-derived gamma-ketoaldehydes and protect against H2O2-mediated cytotoxicity. Biochemistry 45, 15756-15767 (2006).
16. Leopold J A. Antioxidants and coronary artery disease: from pathophysiology to preventive therapy. Coron Artery Dis 26, 176-183 (2015).
17. Roberts L J, 2nd, et al. The relationship between dose of vitamin E and suppression of oxidative stress in humans. Free Radic Biol Med 43, 1388-1393 (2007).
18. Amarnath V, Amarnath K, Amarnath K, Davies S, Roberts L J, 2nd. Pyridoxamine: an extremely potent scavenger of 1,4-dicarbonyls. Chem Res Toxicol 17, 410-415 (2004).
19. Nakajima T, et al. Selective gamma-ketoaldehyde scavengers protect Nav1.5 from oxidant-induced inactivation. J Mol Cell Cardiol 48, 352-359 (2010).
20. Amarnath V, Amarnath K. Scavenging 4-Oxo-2-nonenal. Chem Res Toxicol 28, 1888-1890 (2015).
21. Zagol-Ikapitte I, Amarnath V, Bala M, Roberts L J, 2nd, Oates J A, Boutaud O. Characterization of scavengers of gamma-ketoaldehydes that do not inhibit prostaglandin biosynthesis. Chem Res Toxicol 23, 240-250 (2010).
22. Zagol-Ikapitte I, et al. Determination of the Pharmacokinetics and Oral Bioavailability of Salicylamine, a Potent gamma-Ketoaldehyde Scavenger, by LC/MS/MS. Pharmaceutics 2, 18-29 (2010).
23. Sidorova T N, et al. Reactive gamma-ketoaldehydes promote protein misfolding and preamyloid oligomer formation in rapidly-activated atrial cells. J Mol Cell Cardiol 79, 295-302 (2015).
24. Pitchford L M, et al. First-in-human study assessing safety, tolerability, and pharmacokinetics of 2-hydroxybenzylamine acetate, a selective dicarbonyl electrophile scavenger, in healthy volunteers. BMC Pharmacol Toxicol 20, 1 (2019).
25. Zhang L, Wu T, Olatunji O J, Tang J, Wei Y, Ouyang Z. N(6)-(2-hydroxyethyl)-adenosine from Cordyceps cicadae attenuates hydrogen peroxide induced oxidative toxicity in PC12 cells. Metab Brain Dis, (2019).
26. Yang C F, Shen H M, Ong C N. Protective effect of ebselen against hydrogen peroxide-induced cytotoxicity and DNA damage in HepG2 cells. Biochem Pharmacol 57, 273-279 (1999).
27. Sanda G M, Deleanu M, Toma L, Stancu C S, Simionescu M, Sima A V. Oxidized LDL-Exposed Human Macrophages Display Increased MMP-9 Expression and Secretion Mediated by Endoplasmic Reticulum Stress. J Cell Biochem 118, 661-669 (2017).
28. Bae Y S, et al. Macrophages generate reactive oxygen species in response to minimally oxidized low-density lipoprotein: toll-like receptor 4- and spleen tyrosine kinase-dependent activation of NADPH oxidase 2. Circ Res 104, 210-218, 221p following 218 (2009).
29. Lara-Guzman O J, et al. Oxidized LDL triggers changes in oxidative stress and inflammatory biomarkers in human macrophages. Redox Biol 15, 1-11 (2018).
30. Tao H, et al. Macrophage SR-BI mediates efferocytosis via Src/PI3K/Rac1 signaling and reduces atherosclerotic lesion necrosis. J Lipid Res 56, 1449-1460 (2015).
31. Shao B, et al. Modifying apolipoprotein A-I by malondialdehyde, but not by an array of other reactive carbonyls, blocks cholesterol efflux by the ABCA1 pathway. J Biol Chem 285, 18473-18484 (2010).
32. Gesquiere L, Loreau N, Blache D. Impaired cellular cholesterol efflux by oxysterol-enriched high density lipoproteins. Free Radic Biol Med 23, 541-547 (1997).
33. Salmon S, Maziere C, Auclair M, Theron L, Santus R, Maziere J C. Malondialdehyde modification and copper-induced autooxidation of high-density lipoprotein decrease cholesterol efflux from human cultured fibroblasts. Biochim Biophys Acta 1125, 230-235 (1992).
34. Calderon J C, Fernandez A Z, Maria de Jesus Al. [Atherosclerosis, oxidative stress and physical activity. Review]. Invest Clin 49, 397-410 (2008).
35. Davies S S, Zhang L S. Reactive Carbonyl Species Scavengers-Novel Therapeutic Approaches for Chronic Diseases. Curr Pharmacol Rep 3, 51-67 (2017).
36. Forbes J M, et al. Advanced Glycation End Product Interventions Reduce Diabetes-Accelerated Atherosclerosis. Diabetes 53, 1813-1823 (2004).
37. Watson A M D, et al. Delayed intervention with AGE inhibitors attenuates the progression of diabetes-accelerated atherosclerosis in diabetic apolipoprotein E knockout mice. Diabetologia 54, 681-689 (2011).
38. Barski O A, et al. Dietary Carnosine Prevents Early Atherosclerotic Lesion Formation in Apolipoprotein E-Null Mice. Arteriosclerosis, Thrombosis, and Vascular Biology 33, 1162-1170 (2013).
39. Brown B E, et al. Supplementation with carnosine decreases plasma triglycerides and modulates atherosclerotic plaque composition in diabetic apo E(−/−) mice. Atherosclerosis 232, 403-409 (2014).
40. Menini S, lacobini C, Ricci C, Fantauzzi C B, Pugliese G. Protection from diabetes-induced atherosclerosis and renal disease by d-carnosine-octylester: effects of early vs late inhibition of advanced glycation end-products in Apoe-null mice. Diabetologia 58, 845-853 (2015).
41. May-Zhang L S, et al. Modification by isolevuglandins, highly reactive gamma-ketoaldehydes, deleteriously alters high-density lipoprotein structure and function. J Biol Chem 293, 9176-9187 (2018).
42. Fisher E A, Feig J E, Hewing B, Hazen S L, Smith J D. High-density lipoprotein function, dysfunction, and reverse cholesterol transport. Arterioscler Thromb Vasc Biol 32, 2813-2820 (2012).
43. Rohatgi A, et al. HDL cholesterol efflux capacity and incident cardiovascular events. N Engl J Med 371, 2383-2393 (2014).
44. Khera A V, et al. Cholesterol efflux capacity, high-density lipoprotein function, and atherosclerosis. N Engl J Med 364, 127-135 (2011).
45. Bellanger N, et al. Atheroprotective reverse cholesterol transport pathway is defective in familial hypercholesterolemia. Arterioscler Thromb Vasc Biol 31, 1675-1681 (2011).
46. Ogura M, Hori M, Harada-Shiba M. Association Between Cholesterol Efflux Capacity and Atherosclerotic Cardiovascular Disease in Patients With Familial Hypercholesterolemia. Arterioscler Thromb Vasc Biol 36, 181-188 (2016).
47. Takata K, et al. Impact of cigarette smoking cessation on high-density lipoprotein functionality. Circ J 78, 2955-2962 (2014).
48. Amaki T, et al. Circulating malondialdehyde modified LDL is a biochemical risk marker for coronary artery disease. Heart 90, 1211-1213 (2004).
49. Schiopu A, et al. Recombinant antibodies to an oxidized low-density lipoprotein epitope induce rapid regression of atherosclerosis in apobec-1(−/−)/low-density lipoprotein receptor(−/−) mice. J Am Coll Cardiol 50, 2313-2318 (2007).
50. Tsimikas S, et al. Human oxidation-specific antibodies reduce foam cell formation and atherosclerosis progression. J Am Coll Cardiol 58, 1715-1727 (2011).
51. Hjerpe C, Johansson D, Hermansson A, Hansson G K, Zhou X. Dendritic cells pulsed with malondialdehyde modified low density lipoprotein aggravate atherosclerosis in Apoe(−/−) mice. Atherosclerosis 209, 436-441 (2010).
52. Scull C M, Tabas I. Mechanisms of ER stress-induced apoptosis in atherosclerosis. Arterioscler Thromb Vasc Biol 31, 2792-2797 (2011).
53. Bryk D, Olejarz W, Zapolska-Downar D. The role of oxidative stress and NADPH oxidase in the pathogenesis of atherosclerosis. Postepy Hig Med Dosw (Online) 71, 57-68 (2017).
54. Tabas I. Apoptosis and efferocytosis in mouse models of atherosclerosis. Curr Drug Targets 8, 1288-1296 (2007).
55. Ridker P M, et al. Antiinflammatory Therapy with Canakinumab for Atherosclerotic Disease. N Engl J Med 377, 1119-1131 (2017).
56. Que X, et al. Oxidized phospholipids are proinflammatory and proatherogenic in hypercholesterolaemic mice. Nature 558, 301-306 (2018).
57. Bohula E A, et al. Inflammatory and Cholesterol Risk in the FOURIER Trial. Circulation 138, 131-140 (2018).
58. VanderLaan PA, Reardon C A, Thisted R A, Getz G S. VLDL best predicts aortic root atherosclerosis in LDL receptor deficient mice. J Lipid Res 50, 376-385 (2009).
59. Song L, Leung C, Schindler C. Lymphocytes are important in early atherosclerosis. J Clin Invest 108, 251-259 (2001).
60. Smith D D, Tan X, Tawfik O, Milne G, Stechschulte D J, Dileepan K N. Increased aortic atherosclerotic plaque development in female apolipoprotein E-null mice is associated with elevated thromboxane A2 and decreased prostacyclin production. J Physiol Pharmacol 61, 309-316 (2010).
61. Robinet P, Wang Z, Hazen S L, Smith J D. A simple and sensitive enzymatic method for cholesterol quantification in macrophages and foam cells. J Lipid Res 51, 3364-3369 (2010).
62. Paigen B, Morrow A, Holmes P A, Mitchell D, Williams R A. Quantitative assessment of atherosclerotic lesions in mice. Atherosclerosis 68, 231-240 (1987).
63. Linton M F, Atkinson J B, Fazio S. Prevention of atherosclerosis in apolipoprotein E-deficient mice by bone marrow transplantation. Science 267, 1034-1037 (1995).
64. Makowski L, et al. Lack of macrophage fatty-acid-binding protein aP2 protects mice deficient in apolipoprotein E against atherosclerosis. Nat Med 7, 699-705 (2001).
65. Babaev V R, et al. Macrophage E P4 deficiency increases apoptosis and suppresses early atherosclerosis. Cell Metab 8, 492-501 (2008).
66. Hartig S M. Basic image analysis and manipulation in ImageJ. Curr Protoc Mol Biol Chapter 14, Unit14 15 (2013).
67. Livak K J, Schmittgen T D. Analysis of relative gene expression data using real-time quantitative PCR and the 2(−Delta Delta C(T)) Method. Methods 25, 402-408 (2001).
68. Mohebati A, et al. Effect of zileuton and celecoxib on urinary LTE4 and PGE-M levels in smokers. Cancer Prev Res (Phila) 6, 646-655 (2013).
69. Yermalitsky V N, et al. Simplified LC/MS assay for the measurement of isolevuglandin protein adducts in plasma and tissue samples. Anal Biochem 566, 89-101 (2019).
70. Boutaud O, et al. Levuglandinyl adducts of proteins are formed via a prostaglandin H2 synthase-dependent pathway after platelet activation. J Biol Chem 278, 16926-16928 (2003).
71. Zagol-Ikapite I, et al. Modification of platelet proteins by malondialdehyde: prevention by dicarbonyl scavengers. J Lipid Res 56, 2196-2205 (2015).

It will be understood that various details of the presently disclosed subject matter can be changed without departing from the scope of the subject matter disclosed herein. Furthermore, the foregoing description is for the purpose of illustration only, and not for the purpose of limitation.

We claim:

1. A method of treating familial hypercholesterolemia accelerated atherosclerosis in a subject in need thereof, comprising administering an effective amount of a compound selected from the following formula: 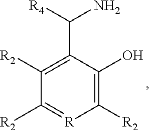
wherein:
R is C—R₂;
each R₂ is independent and chosen from H, substituted or unsubstituted alkyl, halogen, alkyl, substituted or unsubstituted alkoxy, hydroxyl, nitro;
R₄ is H, 2H, substituted or unsubstituted alkyl, carboxyl; and pharmaceutically acceptable salts thereof.

2. The method of claim 1, wherein the subject is diagnosed with familial hypercholesterolemia.

3. The method of claim 1, wherein the compound is selected from the following formula: 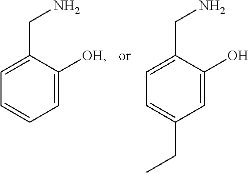
or a pharmaceutically acceptable salt thereof.

4. The method of claim 1, wherein the compound is 2-hydroxybenzylamine, ethyl-2-hydroxybenzylamine, or methyl-2-hydroxybenzylamine.

5. The method of claim 1, wherein the compound is 2-hydroxybenzylamine.

6. The method of claim 1, wherein the compound is selected from the following formula: 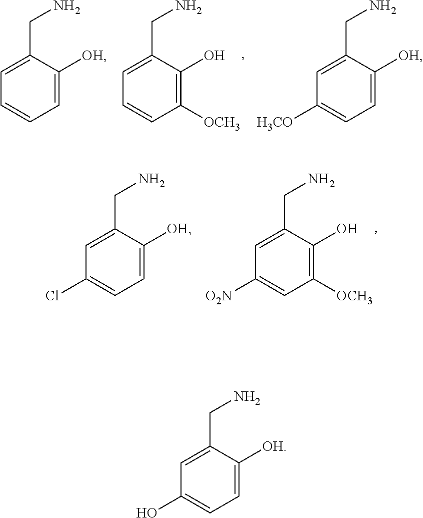
or a pharmaceutically acceptable salt thereof.

7. The method of claim 1, wherein the compound is chosen from: 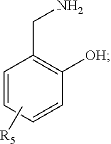
wherein R₅ is H, —CH₃, —CH₂CH₃, —CH(CH₃)—CH₃.

8. A method of reducing MDA- and IsoLG-lysyl content in atherosclerotic aortas in a subject in need thereof, comprising administering an effective dicarbonyl scavenging amount of a compound may be selected from the following formula: 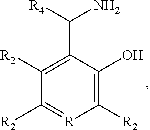
wherein:
R is C—R₂;
each R₂ is independent and chosen from H, substituted or unsubstituted alkyl, halogen, alkyl, substituted or unsubstituted alkoxy, hydroxyl, nitro;
R₄ is H, 2H, substituted or unsubstituted alkyl, carboxyl; and pharmaceutically acceptable salts thereof.

9. The method of claim 8, wherein the subject is diagnosed with familial hypercholesterolemia.

10. A method of treating atherosclerosis in a subject in need thereof, comprising administering an effective dicarbonyl scavenging amount of a compound of the following formula: 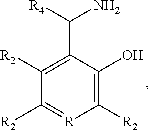
wherein:
R is C—R₂;
each R₂ is independent and chosen from H, substituted or unsubstituted alkyl, halogen, alkyl, substituted or unsubstituted alkoxy, hydroxyl, nitro;
R₄ is H, 2H, substituted or unsubstituted alkyl, carboxyl; and pharmaceutically acceptable salts thereof;
and co-administering a drug with a known side effect of treating atherosclerosis.

11. The method of claim 10, wherein the subject is diagnosed with familial hypercholesterolemia.
